(19) United States
(12) Patent Application Publication
(10) Pub. No.: US 2019/0046584 A1
(43) Pub. Date: Feb. 14, 2019

(54) IMPROVED PREPARATIONS OF ADULT LIVER PROGENITOR CELLS

(71) Applicant: UNIVERSITÉ CATHOLIQUE DE LOUVAIN, Louvain-la-Neuve (BE)

(72) Inventors: Catherine LOMBARD, Louvain-la-Neuve (BE); Pierre-Edouard DOLLET, Louvain-la-Neuve (BE); Etienne SOKAL, Louvain-la-Neuve (BE); Mustapha NAJIMI, Louvain-la-Neuve (BE)

(21) Appl. No.: 16/079,694

(22) PCT Filed: Mar. 2, 2017
(86) PCT No.: PCT/EP2017/054859

§ 371 Date: Aug. 24, 2018

(30) Foreign Application Priority Data
Mar. 2, 2016 (EP) 16158327.3

(51) Int. Cl. A61K 35/407; G01N 33/50; A61P 1/16; A61P 7/02; A61P 7/04

(52) U.S. Cl. CPC A61K 35/407; G01N 33/5067; A61P 7/04; A61P 7/02; A61P 1/16

(57) ABSTRACT

Preparations of adult liver progenitor cells (called HHALPCs) have been manufactured from different human donors and characterized by using cell surface markers that allow identifying HHALPCs preparations and/or the methods for producing them that are most suitable for cell therapy, in particular for treating liver diseases or inherited blood coagulation disorders.

### TECHNICAL FIELD

The present Invention relates to adult liver progenitor cells that are generated using primary liver cells and their use for the medical management of liver diseases, inherited blood coagulation disorders or for screening compounds of medical interest.

### FIELD OF THE INVENTION

Liver is a key organ in the regulation of body homeostasis and is the site of many vital metabolic pathways. Impairment of only one protein within a complex metabolic pathway could be highly deleterious. The large presence of important liver enzymes substantially increases the risk occurrence of diverse liver diseases. Current treatments, and long-term management, are not efficient enough. Orthotopic liver transplantation (OLT) is highly intrusive, irreversible, limited by shortage of donor grafts and demands state-of-art surgery. Liver cell transplantation (LCT) may exert only short-to-medium term efficacy due to the quality of hepatocyte preparations. Further improvements in tolerance towards cryopreservation, permanent engraftment, liver regeneration, and high functionality of the infused cells, would be a major breakthrough (Christ B et al., 2015; Berardis S et al., 2015; Forbes S et al., 2015; Ibars E et al., 2016).

This improvement could be brought by the use of stem or progenitor cells, in particular liver progenitor cells that have been identified in the literature using liver tissues from different organisms, as well as in foetal or adult liver tissues (Schmelzer E et al., 2007; Sahin M B et al., 2008; Azuma H et al., 2003; Herrera M B et al., 2006; Najimi M et al., 2007; Darwiche H and Petersen B E, 2010; Shiojiri N and Nitou M, 2012; Tanaka M and Miyajima A, 2012). Such cells can provide, following in vitro exposure to hepatogenic stimuli and/or after in vivo administration, cells with morphological and functional features typically associated to hepatic differentiation such as phase I/II enzymatic activities.

These liver progenitor cells or hepatocyte-like cells that are generated from them can be used in cellular transplantation as well as for drug testing in the development of new drugs since they represent a surrogate for primary human hepatocytes in drug metabolism and pharmacological or toxicological in vitro screening (Dan Y Y, 2012; Hook L A, 2012). However, it is currently not possible to determine which of the liver progenitor cells so far identified are those more appropriate for therapy of a given disease or use, mainly due to the variability in methods used to produce and characterize such cells for evaluating their potentially therapeutic effects in vivo and the consequent pharmaceutical uses.

The activities, expansion, migration, engraftment, immunogenicity, and differentiation of mesenchymal stem cells in general, such as adult liver progenitor cells having mesenchymal features, depends on specific surface proteins and their immunological profile (Berardis S et al., 2014; Sana G et al., 2014; Najar M et al., 2013; Raicevic G et al., 2015), in particular by obtaining specific cell sub-populations obtained from different donors and/or production process.

However, specific combinations of hepatic markers, mesenchymal markers, tetraspanins, adhesion markers, cell surface receptors, and other categories of markers, have not been used for identifying liver progenitor cells (or mesenchymal stromal cells of liver origin) from different human donors that are produced in cell culture for pharmaceutical uses, i.e. in GMP (Good Manufacturing Practices) conditions. Indeed, the industrial manufacturing of liver progenitor cells for clinical use requires identifying additional, reliable criteria that allow characterizing their quality throughout the process for selecting donors, the cell production and formulation, and/or patients' selection and consequently their efficient pharmaceutical preparation and use.

### SUMMARY OF THE INVENTION

The present Invention is based on the observation that specific cell culture conditions allow obtaining novel adult liver progenitor cell populations with specific marker profile and improved biological features from different human donors. Such cell populations can be used for producing cell-based pharmaceutical compositions (or conditioned media from the corresponding cell cultures) in GMP conditions which can be used within compositions, such as pharmaceutical compositions, in particular for the treatment of liver diseases, inherited blood coagulation disorders, and other human diseases.

These cell preparations represent a cell population that have a marker profile (in particular the expression and the exposure of cell surface proteins) that characterize them as being different from those identified in previously described adult liver progenitor cell populations that are isolated or otherwise produced from human donors in non-GMP conditions such as the adult liver progenitor cells identified in the literature such as ADHLSC Cells (Najimi M et al., 2007; Khuu D N et al., 2011; Scheers I et al., 2012; Berardis S et al., 2014; Maerckx C et al., 2014). Such additional surface markers can provide relevant criteria for producing pharmaceutical preparations with improved viability, proliferation, storage, and/or functional features, especially when their determination is combined with the evaluation of biological activities, including those relevant for specific pharmaceutical compositions and uses of these cells.

In addition, some of these cell surface markers can provide relevant criteria for either characterizing the donors' liver cells that are intended to be used for preparing desired cell populations in GMP conditions (prior to or during manufacturing), or selecting the patients that can be treated with such cell preparations.

A main embodiment of the invention comprises adult liver progenitor cells (named HHALPCs) that can be provided as cell population by means of pharmaceutical manufacturing process under GMP requirements, as well as cell preparations and pharmaceutical compositions comprising them. These cells and cell populations present a combination of protein markers that can be identified on their surface, in particular said cell is measured positive for:
(a) The mesenchymal or pluripotent markers CD13, CD73, CD90, and CD105;
(b) The adhesion markers CD29, CD44, CD47, CD49b, CD49c, CD49e, and CD147;
(c) The tetraspanins CD9, CD63, CD81, and CD151; and
(d) CD98, CD140b, and β2-microglobulin.

These cell populations can be further defined by being measured positive for:
(a) At least one marker selected from adhesion markers CD54, CD164, CD165, and CD166; and/or
(b) At least one marker selected from CD46, CD55, CD59, and CD95.

This cell and related cell populations can be characterized across donors and/or manufacturing processes by a series of cell markers that can be positive or negative. For example, the cell is measured positive for at least one marker selected from CD26, CD49a, CD49d, CD58, CD61, CD71, CD142, CD146, CD201, CD340, and HLA-A/-B/-C.

Alternatively, the cell is measured negative for at least one marker selected from
(a) CD26, CD49a, CD49d, CD58, CD61, CD71, CD142, CD146, CD201, CD340, and HLA-A/-B/-C; and/or
(b) One or more of CD45, CD117, CD34, and HLA-DR.

HHALPCs can be then further measured positive for a series of other markers and activities that are determined as being secreted, on the cell surface, intracellularly, or otherwise expressed by HHALPCs, including:
(a) Positive for at least one hepatic marker selected from albumin, HNF-4, and CYP3A4;
(b) Positive for at least one mesenchymal marker selected from Vimentin, α-smooth muscle actin (ASMA);
(c) Negative for cytokeratin-19 (CK-19).

HHALPCs can be characterized in any functional and technical combination of the above embodiments for positive and negative markers, such as cell and cell populations that are:
(a) Positive for CD13, CD73, CD90, CD105, CD29, CD44, CD47, CD49b, CD49c, CD49e, CD147, CD9, CD63, CD81, CD151, CD98, CD140b, 32-microglobulin, CD54, CD164, CD165, CD166, CD46, CD55, CD59, CD95, Albumin, and Vimentin; and
(b) Negative for CD45, CD117, CD34, and HLA-DR, and Cytokeratin-19.

The cells and cell populations include cells that, before and/or after differentiation in vitro (as well as after administration in animal models and/or in human subjects) present cell type-specific features, in particular functional and expression features of liver cells, preferably hepatocytes. Such liver-specific activities include biological activity related to human CYP450 enzymes, detoxification, bilirubin conjugation, alpha-1-antitrypsin secretion, albumin secretion, secretion of blood coagulation factors, bile production, thrombopoietin production, angiotensinogen production, conversion of ammonia to urea, cholesterol synthesis, glycogenolysis, glycogenesis, and/or lipogenesis.

HHALPCs can be provided as isolated cell populations that comprise cells presenting the biological activity, the markers, and/or functional features listed above in a large majority (e.g. for instance at least 60%, at least 70%, at least 80%, at least 90%, at least 95%, or at least 99%). In a preferred embodiment, a HHALPC Progeny is a cell population that comprises at least 60%, or between 60% and 99% or between 70% and 90% of cells that are measured positive and, optionally, negative for the markers as indicated above and that may be associated to features related to useful for HHALPCs manufacturing and/or uses.

HHALPCs of any of the above embodiments can be used for providing additional, isolated cell populations, collectively grouped under the name of HHALPC Progeny, comprising HHALPCs as defined above that are obtained by passaging them in GMP cell culture conditions. In particular, HHALPC Progeny results from the maintenance, proliferation, and/or differentiation of HHALPCs in cell culture conditions (or following implantation in humans or in an animal model), as required for the desired use. HHALPC Progeny can be provided as adherent cells or forming three-dimensional cell clusters (in suspension, within scaffolds, or comprised in other structures that may allow providing cells presenting improved storage, formulations, and/or activities) that are passaged no more than 2, no more than 3, no more than 4, or no more than 5 times in culture. Moreover, such cell population can be further differentiated into cells presenting liver-specific biological activities, in vitro and/or and in vivo.

HHALPCs and HHALPC Progeny can be also modified by means of one or more chemical agents, cell culture medium, growth factors, and/or nucleic acids vectors for any in vivo or in vitro use that requires appropriately adding or eliminating any properties of such cells.

The methods for obtaining HHALPCs and HHALPC Progeny are established using primary liver cells of human origin (fresh or cryopreserved) in GMP conditions, that is with equipment, cell culture containers and biological materials as required for cell therapy in humans. The development of methods for producing HHALPCs involves measuring the positivity (and, optionally, also negativity) for specific combinations of markers as defined above. Then, depending on the desired use of HHALPCs and HHALPC Progeny, the cells that are obtained or obtainable by this method can be maintained in cell culture conditions allowing their proliferation as adherent cells, cell suspensions, or, by applying specific conditions for maintaining them, as hepatocyte-like or hepato-active cells, using commercially available low adherence container (in the form of plates or U-shaped wells), in cell culture stacks, micro-carriers, or in a bioreactor and characterized according to their functional and/or antigenic features as defined above.

Biological materials that are obtained when generating HHALPCs or an HHALPC Progeny can be further used for identifying biological entities that may have specific uses, in particular distinct medical applications for treating a condition that may benefit from HHALPCs engraftment in a human tissue. These biological materials include not only HHALPCs in general but also sub-population, cell lines, and fraction thereof that present specific features (e.g. protein- or nucleic acid-based markers, biological activities, and/or morphology) but also any other entity that is obtained when producing HHALPCs or HHALPC Progeny. Biological materials of the invention include, for example, conditioned cell culture media (e.g. in form of cell culture supernatant) and fractions of these media that may contain proteins, metabolites, membrane vesicles, antigens, and/or nucleic acids that, together or not with other features characterizing the cells themselves (e.g. cell surface antigen or enzymatic activities), can be identified and used as markers for detecting cells of medical interest or as compounds or biological products that present activities or distribution of medical interest, in particular for liver diseases.

HHALPCs, HHALPC Progeny, biological materials that are obtained when generating HHALPCs or an HHALPC Progeny, and compositions comprising such cells or biological materials (“HHALPC Products”, collectively), can be useful for a large number of methods and uses, either in vivo or in vitro. Preferably, HHALPCs can be used in accordance to the disclosure of WO2007071339 and literature on ADHLSC Cells, on adult liver progenitor/stem cells in general, or in the Examples.

An HHALPC Product can be used for treating diseases (e.g. liver diseases) and for establishing methods and biological assays that require cells presenting biological features (such as metabolic or enzymatic activities, or an antigenic profile) as similar as possible to those observed for primary hepatocytes for the desired period of time, once they are differentiated either in vivo or in vitro. Preferred HHALPC Products are an HHALPC Progeny, a biological material that is obtained when generating HHALPC Progeny, and a composition comprising either HHALPC Progeny or such biological material. More preferably, an HHALPC Product is an HHALPC Progeny or a composition comprising an HHALPC Progeny that is formulated for medical use (i.e. as a cell therapy product for intrahepatic, intrasplenic, intravenous, or intra-articular administration).

In particular, an HHALPC Product can be used for in vivo administration (in humans or in animals, such as in animal models), for example in the form of a pharmaceutical composition comprising such cells, for treating an inherited Blood Coagulation Disorder or a liver disease (such as an inborn error of liver metabolism, progressive familial intrahepatic cholestasis type 1/2/3, alpha 1-Antitrypsin Deficiency, defect of liver cell transporters, Porphyria, fatty liver or other fibrotic liver disease, primary biliary cirrhosis, sclerosing cholangitis, liver degenerative disease, non-alcoholic steatohepatitis, liver fibrosis, and acute-on-chronic liver failure). HHALPCs Products may be provided in the form of a pharmaceutical composition comprising them, for treating human diseases, in particular diseases that require enzymatic, immunomodulatory, or other effects within liver or in other tissues, with respect to functions related to protein that are secreted by liver cells and having effects on liver or other tissues and organs (such as in blood, articulations, bone marrow, spleen, or intestines).

These pharmaceutical compositions can be provided as HHALPC Products that are combined with a support (e.g. a matrix, a capsule, a scaffold, or a device) and/or a solution (e.g. cell culture medium or buffer) that is appropriate for the desired method of treatment, administration, use and/or storage, as well as in the preferred means for providing such pharmaceutical compositions (e.g. within a kit). Other agents of biological (e.g. an antibody or a growth factor) or chemical origin (e.g. drugs, preserving or labelling compounds) that may provide any further effect can be also combined in such compositions.

A method for preventing and/or treating a disease comprises administering an HHALPC Product, such as HHALPCs or a given HHALPC Progeny, and preferably within a composition, to a subject in need thereof. In particular, a method of treating a disease (e.g. a liver disease) in a patient in need thereof comprises administering an effective amount of an HHALPC Product to the patient.

The administration or the therapeutic use of an HHALPC Product may comprise the administration or use of another product (which may be, for example a drug, a therapeutic agent, another cell type, or other biological material). An HHALPC Product may be used in (or for use in) a method of treatment as described herein, wherein the patient is also administered such another product as part of the method. The other product may be administered in combination with the HHALPC Product, for example as part of the same composition, or separately, in a simultaneous or sequential manner (and in any order). The other product may have effects that are compatible, additive or even synergistic, with the effects (in particular with the therapeutic effects) of an HHALPC Product, such an HHALPC Progeny or a conditioned cell culture media obtained from an HHALPC Progeny.

An HHALPC Product can also be used for in vitro studies, in particular for pharmacological studies for evaluating the efficacy, metabolism, stability and/or toxicity of one or more exogenous components such as a biological product (such a protein, a nucleic acid, lipids, or a sugar) or a chemical compound (organic or inorganic, including salts or metals). This approach may be used also for studying effects of other cells (such as bacteria or other cells, preferably of human origin) on an HHALPC Product, as well as evaluating the infection and/or the replication of liver-specific viruses (e.g. hepatitis viruses) or parasites (like those Plasmodium species, in connection to the study of malaria and anti-malarial drugs) that can be later purified or otherwise detected.

Thus, the present invention also provides methods for evaluating the efficacy, the metabolism, the stability, and/or the toxicity of one or more exogenous components (i.e. an organic or inorganic compound), either in vitro or in vivo, said method comprising:
(a) providing an HHALPC Product;
(b) exposing said HHALPC Product to one or more compounds (selected from chemical compounds, proteins, nucleic acids, lipids, sugars, metals, salts, viruses, bacteria, and cells); and
(c) detecting the effects of said one or more compounds on said HHALPC Product and/or detecting the presence, localization, or modification of said one or more compounds following the exposure to said HHALPC Product.

This general method can include in some embodiments further steps and features that apply to specific uses and/or technologies. For instance, step (c) as defined above can comprise detecting the effects on cell morphology, on cell viability, on up- or down-regulation of liver-specific or unspecific proteins, and/or on the degradation, aggregation, secretion, internalization, activation, or inhibition of proteins within an HHALPC Product. Furthermore, step (c) as defined above can comprise detecting the internalization of such one or more compounds into, or the physical association with, an HHALPC Product. The HHALPC Product can be also provided to an animal, such as a non-human animal, at step (a), and then one or more compounds is administered to said animal in step (b). Finally, the step (c) comprises detecting the effects of said one or more compounds on said HHALPC Product or on said animal, and/or detecting the presence, localization, or modification of said one or more compounds following the exposure to said HHALPC Product in the animal.

The methods of using HHALPC Products may also involve exposing the cell population, composition, or biological material in step (b), simultaneously or sequentially in any order, to (i) one or more compounds that have an effect cell morphology, cell viability, up- or down-regulation of liver-specific or unspecific proteins, and/or that degrade, aggregate, activate, or inhibit proteins within an HHALPC Product; and (ii) one or more compounds that is intended to block or avoid such effects within the HHALPC Product.

In some embodiments, this method is intended to use any HHALPC Product, and in particular an HHALPC Progeny as a model of hepatic cells for determining if, when exposed to a compound that is pathogenic agent, a further compound that is a candidate drug specifically targeting the pathogenic agent and/or their effects has therapeutic properties since it prevents or blocks any undesirable effect of the pathogenic agent (e.g. viral infection, apoptosis, oncogenic transformation, reduction of liver-specific activities, etc.). In particular, the compound of (i) above that is pathogenic agent, comprises an infectious, tumorigenic, cytotoxic, or genotoxic agent, and the further compounds of (ii) above that is a candidate drug specifically targeting the pathogenic agent and/or their effects, comprises a protein, a nucleic acid, a cell, a virus, or a chemical compound.

The HHALPC Product can also be provided in a kit, for example, for the uses and methods of the applications as described above, including for transferring an HHALPC Product to a clinical institution and providing means for administering it to a patient. This kit can comprise an HHALPC Product and, optionally, further elements that allow using and/or detecting the HHALPC Product and their activities, as well as for using and/or detecting any relevant additional compound. This kit can comprise one or more vials containing an HHALPC Product (e.g. an HHALPC Progeny or a composition comprising HHALPC Progeny) and one or more of the following elements to be selected according to the specific use: devices, disposable materials, solutions, chemical products, biological products, and/or instructions for using the elements of said kit.

The Detailed Description and the Examples provide additional details on the cells, the cell populations, the methods, and on further embodiments of the Invention that are associated to HHALPCs and HHALPC Progeny.

### DESCRIPTION OF FIGURES

FIG. 1: Detection of cell surface proteins for characterizing HHALPCs during GMP production. Cell surface proteins such as CD44, VLA-2 (complex comprising CD29 and CD49b), VLA-3 (comprising CD29 and CD49c), and VLA-5 (complex comprising CD29 and CD49e) are exposed on the surface of HHALPCs (A; peak at 0 in each panel corresponds to the signal of isotype control antibody). Otherwise, CXCR4 (CD184) is detected during the cell culture passages by flow cytometry only following cell permeabilization, suggesting its expression but rapid internalization by HHALPCs.

FIG. 2: In vivo Ureagenesis is measured in the plasma of different HHALPCs-treated patients (each identified with a different symbol) suffering from different Urea Cycle Disorders prior to (baseline) and at 2 later time points (3 and 6 months) after infusions of HHALPCs.

FIG. 3: Detection and therapeutic activity of HHALPCs in a patient suffering from Haemophilia A. A fraction of HHAPLCs were labelled prior to intravenous administration with ¹¹¹In-DTPA and the bio-distribution was followed by Single Photon Emission Computed Tomography (SPECT) imaging. Labelled HHALPCs that are administered intravenously are found concentrating in liver and spleen (A) The signal intensity for the relative distribution of HHALPCs at various sites was found decreasing in the lungs and simultaneously increasing in the liver, when comparing the images acquired at 24, 48, 72 hours and 6 days post infusion (B). When Factor VIII consumption is analysed, the patient's factor VIII baseline requirement was approximately 5000 IU/week with extra doses of 2000 IU administered prior to the infusion (in addition to the baseline during the four-HHALPC injection regimen) but the amount of Factor VIII that the patients required for a normal haemostasis in the following 15 weeks was considerably reduced (C).

FIG. 4 Therapeutic activity of HHALPCs in a patient suffering from ornithine transcarbamylase (OTC) deficiency with a late onset disease presentation. The cell therapy was administered on 4 infusions days (inf. 01-04; one infusion per day), spread over a 8-week period with an interval of two weeks between infusion days. The infusions period was completed between February and March 2016. During this treatment period, the patient was closely followed-up with a medical control at day 1 and day 7 post-infusion. Just after the infusions period, ammonia blood levels were stable during a 2-month period (A). Glutamine blood levels are also normalized in the following months (B).

### DETAILED DESCRIPTION OF THE INVENTION

A main embodiment of the invention comprises HHALPCs and HHALPC Progeny characterized by novel combinations of biological activities and markers that can be identified on their surface and, optionally, intracellularly, and/or secreted in cell culture medium. These features, together with morphological and functional features, were determined in association to the methods for producing HHALPCs and HHALPC Progeny in cell culture conditions, defining the positive (or negative) criteria characterizing such cells. In particular, such method comprises:
(a) Disassociating adult liver or a part thereof to form a population of primary liver cells;
(b) Generating preparations of the primary liver cells of (a);
(c) Culturing the cells comprised in the preparations of (b) onto a support which allows adherence and growth of cells thereto and the emergence of a population of cells;
(d) Passaging the cells of (c) at least once; and
(e) Isolating the cell population that is obtained after passaging of (d) that are positive for the markers identified in the Summary of the Invention.

Concerning Step (a) of the method, the dissociation step involves obtaining adult liver or a part thereof that contains, together with fully differentiated hepatocytes, an amount of primary cells that can be used for producing HHALPCs. The liver primary cells are preferentially isolated from human liver tissues that are obtained from adult liver.

The term “liver” refers to liver organ. The term “part of liver” generally refers to a tissue sample derived from any part of the liver organ, without any limitation as to the quantity of the said part or the region of the liver organ where it originates. Preferably, all cell types present in the liver organ may also be represented in the said part of liver. Quantity of the part of liver may at least in part follow from practical considerations to the need to obtain enough primary liver cells for reasonably practicing the method of the invention. Hence, a part of liver may represent a percentage of the liver organ (e.g. at least 1%, 10%, 20%, 50%, 70%, 90% or more, typically w/w). In other non-limiting examples, a part of liver may be defined by weight (e.g. at least 1 g, 10 g, 100 g, 250 g, 500 g, or more). For example, a part of liver may be a liver lobe, e.g., the right lobe or left lobe, or any segment or tissue sample comprising a sufficient number of cells that is resected during split liver operation or in a liver biopsy.

The term “adult liver” refers to liver of subjects that are post-natal, i.e. any time after birth, preferably full term, and may be, e.g., at least at least 1 day, 1 week, 1 month or more than 1 month of age after birth, or at least 1, 5, 10 years or more. Hence, an “adult liver”, or mature liver, may be found in human subjects who would otherwise be described in the conventional terms of “infant”, “child”, “adolescent”, or “adult”. The liver or part thereof is obtained from a “subject” or “donor”, interchangeably referring to a vertebrate animal, preferably a mammal, more preferably a human. In another embodiment, the adult liver or part thereof may be from a non-human animal subject, preferably a non-human mammal subject (e.g. a rodent or pig).

A donor may be living or dead, as determined by clinically accepted criteria, such as the “heart-lung” criteria (involving an irreversible cessation of circulatory and respiratory functions) or the “brain death” criteria (involving an irreversible cessation of all functions of the entire brain, including the brainstem). Harvesting may involve known procedures such as biopsy, resection or excision. Harvesting of liver tissue from a living human donor may need to be compatible with sustenance of further life of the donor. The liver or part thereof may be obtained from a donor, esp. human donor, who has sustained circulation, e.g., a beating heart, and sustained respiratory functions, e.g., breathing lungs or artificial ventilation. Only a part of liver may typically be removed from a living human donor (e.g., by biopsy or resection), such that an adequate level of normal liver functions is maintained in the donor, as required by legal and ethical norms.

Subject to ethical and legal norms, the donor may need to be or need not be brain dead (e.g., removal of entire liver or portion thereof, which would not be compatible with further survival of a human donor, may be allowed in brain dead human beings). Harvesting of liver or part thereof from such donors is advantageous, since the tissue does not suffer substantial anoxia (lack of oxygenation), which usually results from ischemia (cessation of circulation). At the time of harvesting the tissue may have ceased circulation and/or respiratory functions, with no artificial ventilation. While liver or part thereof from these donors may have suffered at least some degree of anoxia, liver from cadaveric donors can be used for obtaining HHALPCs in cell culture conditions, for instance within about 1, 3, 6, 12, 24 hours or more after the donor's circulation ceased.

The tissues (from surgically resected liver samples or liver biopsies) that are harvested as indicated above may be cooled to about room temperature, or to a temperature lower than room temperature, but usually freezing of the tissue or parts thereof is avoided, esp. where such freezing would result in nucleation or ice crystal growth. For example, the tissue may be kept at any temperature between about 1° C. or about 4° C. and room temperature, and may advantageously be kept at about 4° C., e.g. on ice. The tissue may be cooled for all or part of the ischemic time, i.e., the time after cessation of circulation in the donor. That is, the tissue can be subjected to warm ischemia, cold ischemia, or a combination of warm and cold ischemia. The harvested tissue may be so kept for, e.g., up to 48 hours before processing, preferably for less than 24 hours, e.g., more preferably for less than 12 hours (e.g., less than 6, 3, or 1 hour). The harvested tissue may advantageously be, but needs not to be kept in, e.g., completely or at least partly submerged in, a suitable medium and/or may be but needs not to be perfused with the suitable medium, before further processing of the tissue. A skilled person is able to select a suitable medium that can support the survival of the cells of the tissue during the period before processing.

The method of the invention comprises disassociating adult liver tissue as described above to form a population of primary cells. The term “disassociating” as used herein generally refers to partly or completely disrupting the cellular organization of a tissue or organ, i.e., partly or completely disrupting the association between cells and cellular components of a tissue or organ, to obtain a suspension of cells (a cell population) from the said tissue or organ. The suspension may comprise solitary or single cells, as well as cells physically attached to form clusters or clumps of two or more cells. Disassociating preferably does not cause or causes as small as possible reduction in cell viability. A suitable method for disassociating liver or part thereof to obtain a population (suspension) of primary cells therefrom may be any method well known in the art, including but not limited to, enzymatic digestion, mechanical separation, filtration, centrifugation and combinations thereof. In particular, the method for disassociating liver or part thereof may comprise enzymatic digestion of the liver tissue to release liver cells and/or mechanical disruption or separation of the liver tissue to release liver cells. Small, thin fragments of liver tissues that are obtained by a liver biopsy may be used directly for pursuing cell culture according to the following Step (c) without enzymatic or mechanical disruption.

Methods for disassociating liver or part thereof as above are documented in the literature as the widely used collagenase perfusion technique in two or more steps, which has been variously adapted and modified for performing it with whole livers or segments of liver. The liver tissue is perfused with a divalent cation-free buffer solution, preheated at 37° C., containing a cation-chelating agent (e.g. EDTA or EGTA). Buffer solutions can comprise salt solutions (e.g. HEPES, Williams E medium) or any other balanced salt solution that can also include salts such as sodium Chloride or potassium chloride, among others. This leads to disruption of the desmosomal structures that hold cells together. The tissue is then perfused with the buffer solution containing divalent cation(s), such as Ca²⁺ and Mg²⁺, and matrix-degrading enzymes that act to digest the tissue.

The primary liver cells are usually released by gentle mechanical disruption and/or pressing through filters, to complete the cell dissociation process. Such filters may have sieve sizes that allow passage of cells through about 0.1 mm, 0.25 mm, 0.50 mm, 1 mm or more. A succession of filters with progressively smaller sieve sizes may be used to gradually disassociate the tissue and release cells. The dissociated cells are rinsed with a buffer containing protease inhibitor, serum and/or plasma to inactivate collagenase and other enzymes used in the perfusion process, and then separated from the mixture by pelleting them with low speed centrifugation (e.g. at between 10×g and 500×g). Most of, if not all, viable cells can be pelleted, while dead cells and cell debris are substantially eliminated in the supernatant and subsequently are washed with ice-cold buffer solution to purify the cell suspension. The number and quality of the primary liver cells can vary depending on the quality of the tissue, the compositions of different solutions that are used, and the type and concentration of enzyme. The enzyme is frequently collagenase but also pronase, trypsin, hyaluronidase, thermolysin, and combinations thereof can be used. Collagenase may consist of a poorly purified blend of enzymes and/or exhibit protease activity, which may cause unwanted reactions affecting the quality and quantity of viable cells that can in turn be avoided by selecting enzyme preparations of sufficient purity and quality. Other methods of harvesting primary liver cells may exclude enzymatic digestion techniques and may involve perfusing liver with solutions containing sucrose followed by mechanical disruption.

Concerning Step (b) of the method, the preparation of liver primary cells that is obtained following the disassociation of liver tissue may typically be a heterogeneous population of primary liver cells, comprising cells belonging to any liver-constituting cell types, including progenitor or stem cells, that may have been present in liver parenchyma and or in non-parenchyma thereof. Exemplary liver-constituting cell types include hepatocytes, cholangiocytes, Kupffer cells, hepatic stellate cells, and liver endothelial cells, in addition to stem or progenitor cells that may be present or derived in cell culture conditions from the liver tissue.

The term “hepatocyte” encompasses epithelial, parenchymal liver cells, including but not limited to hepatocytes of different sizes or ploidy (e.g., diploid, tetraploid, octaploid).

The term “primary cell” includes cells present in a suspension of cells obtained from a tissue or organ of a subject, e.g. liver, by disassociating cells present in such explanted tissue or organ with appropriate techniques.

The methods of the Invention may preferably start from a cell population representative of most, if not all, liver cell types at the scope of obtaining the desired adult liver progenitor cells in cell culture conditions. A suitable starting cell population for obtaining HHALPCs may comprise hepatocytes in different proportions (0.1%, 1%, 10%, or more of total cells), according to the method of disassociating liver and/or any methods for fractioning or enriching the initial preparation for hepatocytes and/or other cell types on the basis of physical properties (dimension, morphology), viability, cell culture conditions, or cell surface marker expression by applying any suitable techniques.

The population of primary cells as defined and obtained herein by disassociating liver (or part of it) can be used immediately for establishing cell cultures as fresh primary liver cells or, preferably, stored as cryopreserved preparations of primary liver cells using common technologies for their long-term preservation. Indeed, the use of cryopreserved cell preparations appears having a positive effect on the efficiency with which HHALPCs and HHALPC Progeny are later produced in cell culture. Cells in these samples may be frozen in a cell culture medium or a solution for preserving cells or organs (e. g. Viaspan, Cryostor, Celsior) that is supplemented or not with other compounds such as growth factors, serum, buffer solutions, Glucose, Albumin, ethylene glycol, sucrose, dextrose, DMSO or any other cryoprotectant. Each cryopreserved preparation may contain at least 10³, 10⁴, 10⁵, 10⁶, 10⁷, 10⁸ cells or more per cryovial or bag, at scope of producing and isolating higher amount of HHALPCs in cell culture conditions after appropriately thawing the sample and, if needed, washing the cells with appropriate buffer or cell culture medium for eliminating residual cell culture medium or a solution for preserving cells or organs.

Concerning Step (c) of the method, the preparation of liver primary cells (as a cell suspension or as fragments of liver tissues that are obtained by a liver biopsy) can be cultured directly onto a fully synthetic support (e.g. plastic or any polymeric substance) or a synthetic support pre-coated with feeder cells, protein extracts, or any other material of biological origin that allow the adherence and the proliferation of similar primary cells and the emergence of a population of adult liver progenitor cells having the desired markers, such markers being identified preferably at the level of protein, by means of immunohistochemistry, flow cytometry, or other antibody-based technique.

Preferably cells from the primary cell population that have adhered to the said substrate, are cultured for at least 2 days or more, preferably 7 days, at least 10, or at least 12 days. More preferably, the cells from the primary cell population are cultured within 7 and 12 days, to obtain a population of adherent cells that is sufficiently enriched for viable primary cells that can provide HHALPCs.

The term “culturing” broadly refers to conditions for the maintenance and/or growth of cells, and in particular of HHALPC and/or of HHALPC Progeny in cell culture. Elements such as the support where cells are cultured and allowing cell adhesion (or, when needed, allowing growth of cell clusters in suspension), composition of cell culture medium, density at which the cells are seeded and maintained, the O₂ and CO₂ concentration, may be adapted for culturing HHALPCs and HHALPC Progeny, as detailed below and in the Examples.

The term “liver progenitor cell” refers to an unspecialized and proliferation-competent cell that is produced by culturing cells that are isolated from liver and which or the progeny of which can give rise to at least one relatively more specialized cell type. A liver progenitor cell give rise to descendants that can differentiate along one or more lineages to produce increasingly more specialized cells (but preferably hepatocytes or hepato-active cells), wherein such descendants may themselves be progenitor cells, or even to produce terminally differentiated liver cells (e.g. fully specialized cells, in particular cells presenting morphological and functional features similar to those of human hepatocytes).

HHALPCs are adult liver progenitor cells generated in GMP conditions that can be further characterized by technologies that allow detecting relevant markers already at this stage (that is, before passaging cells as indicated in step (d)) and that were initially characterized at a later stage, as described below at step (e). Among the technologies for identifying such markers and measuring them as being positive or negative, Western Blot, Flow Cytometry immunocytochemistry or analysis of cell culture media are preferred since allowing marker detection at protein level even with the low amount of HHALPCs that are available at this step.

HHALPCs emerge from primary population of liver cells that is plated onto a substrate that allows the adherence of cells within an in vitro environment capable of promoting survival and/or growth of such cells. This environment may prevent an undesired exchange of matter between the said environment (i.e. the cell culture container) and the surroundings (e.g. by avoiding contamination of the laboratory environment), while it can allow continuous or intermittent exchange of other, useful, components between culture vessels (e.g. by an occasional or continuous exchange of a part or all of the culture medium and of gases).

The culture vessels can be cell culture flasks, bottles, well plates, multi-tray cell stacks, bioreactors and dishes of various formats but displaying one or more substrate surfaces compatible with cell adhesion, such that the plated cells can contact this substrate to be maintained adherent cell cultures. In general, a substrate which allows adherence of cells thereto may be any substantially hydrophilic substrate, being glass or a synthetic polymeric material (such as polycarbonates, polystyrenes, polyorthoesters, polyphosphazenes, polyphosphates, polyesters, nylons or mixtures thereof) that are generally shaped and treated in order to provide hydrophilic substrate surfaces and thereby enhance the likelihood of effective cell attachment (as shown in the Examples by using CellBind commercial materials). Surface treatment may take the form of a surface coating, or may involve generating chemical groups on the polymer surface that have a general affinity for water or otherwise exhibit sufficient polarity to permit stable adsorption to another polar group. These functional groups lead to hydrophilicity and/or an increase in surface oxygen and are properties recognized to enhance cell growth on so modified substrate surfaces. Such chemical groups may include groups such as amine, amide, carbonyl, carboxylate, ester, hydroxyl, or sulfhydryl groups that can be also introduced by treating them with specific wave frequency-based technologies.

Cell adhesion can be facilitated by coating the treated plastic surfaces with a layer of a suitable matrix. The coating may involve suitable polycations (e.g., polyomithine or polylysine) or, preferably, one or more components of extracellular matrix that can be provided for GMP manufacturing: laminins, non-/fibrous collagens (preferably collagen type 1), glycosaminoglycans (e.g., heparin or heparan sulphate) or proteins such as fibronectin, gelatine, vitronectin, elastin, tenascin, aggrecan, agrin, bone sialoprotein, cartilage matrix protein, fibrinogen, mucins, or cell adhesion molecules including cadherins or connexins, alone or in various combinations. Preferred examples may include collagen compositions, comprising or not other extracellular matrix components). Alternatively, synthetic peptides that are fragments or otherwise derived from the proteins listed above, gels, molecular scaffolds and other three-dimensional structures that are formed from synthetic and/or biological materials can be used in this scope.

The primary cell suspension may be contacted with the adherent surface for a period of time (e.g. at least 2, 4, 6, 12, 24 hours, or more) that is sufficient for allowing the primary liver cell populations to attach to adherent substrate, before removing any non-adherent matter from the culture system (e.g., non-viable or dead cells and cell debris) by discarding medium from the culture system and optionally washing, once or repeatedly, the adherent cells. Then, the culture system is provided with any suitable medium or isotonic buffer (e.g., PBS). Hereby, cells from the primary liver cell population, which have adhered to the surface, are selected for further culturing and may be counted in order to evaluate the plating density that may be expressed as number of cells plated per cm² of the said surface (e.g. between 10 and 10⁵ cells/cm²).

The preparation of primary cells, directly at plating or after washing the cells, is maintained in a liquid medium, which supports their survival and/or growth of the cells. The medium may be added to the system before, together with or after the introduction of the cells thereto. The medium may be fresh (i.e., not previously used for culturing of cells) or may comprise at least a fraction which has been conditioned by prior culturing cells of liver origin (or of any other origin) therein. In particular, the medium may be any suitable culture medium for culturing liver progenitor cells as described in the literature and it may be regularly exchanged (e.g., each hour, 3 hours, 12 hours, 24 hours or more) with a fresh medium presenting the same or different features (e.g. composition, pH, or oxidative status). The whole volume of the medium may be changed or, alternatively, only part of the medium may be changed, such that a fraction of the medium conditioned by the previous culturing of the cells is retained. Alternatively, the medium is not exchanged until the cells are transferred into another culture vessel, prolonging the culture of the cells in a way that most of the cells not of interest (e.g. hepatocytes and other fully differentiated cells of liver origin) are detached and die, and fresh medium may be simply added regularly.

The adherent, primary cells are cultured in the presence of a liquid culture medium for growing adherent cells that, in accordance to GMP requirements, is based on defined chemical media with (or without) addition of bovine, human or other animal serum. These media, that can be supplemented with appropriate mixture of organic or inorganic compounds may, besides providing nutrients and/or growth promoters, also promote the growth/adherence or the elimination/detachment of specific cell types.

Basal media formulations (available, e.g., from the American Type Culture Collection, ATCC; or from Invitrogen, Carlsbad, Calif.) can be used to culture the primary cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's MB 752/1 or Williams Medium E, and modifications and/or combinations thereof. Compositions of these basal media and criteria to adapt concentrations of media and/or media supplements as necessary for the cells cultured are generally known. A preferred basal medium formulation may be one of those available commercially such as Williams Medium E, IMDM or DMEM, which are reported to sustain in vitro culture of adult liver cells, and including a mixture of growth factors for their appropriate growth, proliferation, maintenance of desired markers and/or biological activity, or long-term storage.

Such basal media formulations contain ingredients necessary for mammal cell development, which are known per se such as inorganic salts (in particular salts containing Na, K, Mg, Ca, CI, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate). Additional supplements can be used to supply the cells with the necessary trace elements and substances for optimal growth and expansion. Such supplements include insulin, transferrin, selenium salts, and combinations thereof. These components can be included in a salt solution such as Hanks' Balanced Salt Solution (HBSS), Earle's Salt Solution. Further antioxidant supplements may be added, e.g. β-mercaptoethanol. While many basal media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds. Most importantly, cell culture media can be complemented with mammalian plasma or sera that contain cellular factors and components that are necessary for cell viability and expansion and that, under certain condition, may be replaced with synthetic components.

The term “serum”, as conventionally defined, is obtained from a sample of whole blood by first allowing clotting to take place in the sample and subsequently separating the so formed clot and cellular components of the blood sample from the liquid component (serum) by an appropriate technique, typically by centrifugation. An inert catalyst, e.g., glass beads or powder, can facilitate clotting. Advantageously, serum can be prepared using serum-separating vessels (SST), which contain the inert catalyst to mammals.

The serum or plasma may be obtained commercially and from an organism of the same species as is the species from which the primary liver cells are obtained. Human serum or plasma may be used for culturing primary human liver cells. Alternatively, the medium comprises bovine serum or plasma, preferably foetal bovine (calf) serum or plasma, more preferably foetal bovine (calf) serum (FCS or FBS). The medium comprises between about 0.5% and about 40% (v/v) of serum or plasma or serum replacement, preferably between about 5% and 20% (v/v), e.g., between about 5% and 15% (v/v), e.g. about 10% (v/v). A medium for culturing human liver cells may comprise a mixture of human plasma or serum, preferably human serum, and bovine plasma or serum, preferably bovine serum.

Prior to storage or use, the plasma or serum can be irradiated (e.g. gamma-irradiated) or heat inactivated. Heat inactivation is used in the art mainly to remove the complement. Heat inactivation typically involves incubating the plasma or serum at 56° C. for 30 to 60 minutes, e.g., 30 minutes, with steady mixing, after which the plasma or serum is allowed to gradually cool to ambient temperature. Optionally, the plasma or serum may also be sterilized prior to storage or use (e.g. by filtration through one or more filters with pore size smaller than 1 μm) or treated in accordance to any applicable regulatory policy for culturing human cells for therapeutic use.

Ordinary components of basal media (before addition of serum or plasma), e.g., in particular, isotonic saline, buffers, inorganic salts, amino acids, carbon sources, vitamins, antioxidants, pH indicators and antibiotics, are not considered growth factors or differentiation factors in the art. On the other hand, serum or plasma is a complex composition possibly comprising one or more such growth factors.

The term “growth factor” as used herein refers to a biologically active substance which influences proliferation, growth, differentiation, survival and/or migration of various cell types, and may effect developmental, morphological and functional changes in an organism, either alone or when modulated by other substances. A growth factor may typically act by binding, as a ligand, to a receptor (e.g., surface or intracellular receptor) present in cells. A growth factor herein may be particularly a proteinaceous entity comprising one or more polypeptide chains. The term “growth factor” encompasses the members of the fibroblast growth factor (FGF) family, bone morphogenic protein (BMP) family, platelet derived growth factor (PDGF) family, transforming growth factor beta (TGF-beta) family, nerve growth factor (NGF) family, the epidermal growth factor (EGF) family, the insulin related growth factor (IGF) family, the hepatocyte growth factor (HGF) family, the interleukin-6 (IL-6) family (e.g. oncostatin M), hematopoietic growth factors (HeGFs), the platelet-derived endothelial cell growth factor (PD-ECGF), angiopoietin, vascular endothelial growth factor (VEGF) family, or glucocorticoids. Where the method is used for human liver cells, the growth factor used in the present method may be a human or recombinant growth factor. The use of human and recombinant growth factors in the present method is preferred since such growth factors are expected to elicit a desirable effect on cellular function

The medium may comprise a combination of serum or plasma with one or more exogenously added growth factors as defined above, preferably at concentrations in which particular growth factors can induce an effect on in vitro cultured cells. For example, the medium may comprise EGF and insulin, or EGF and dexamethasone, or insulin and dexamethasone, or each EGF, insulin and dexamethasone. EGF may be typically used at concentrations between about 0.1 ng/ml and 1 μg/ml and preferably between 1 ng/ml and 100 ng/ml, e.g., at about 25 ng/ml; insulin can be typically used at concentrations between about 0.1 μg/ml and 1 mg/ml and preferably between about 1 μg/ml and 100 μg/ml, e.g., at about 10 μg/ml; dexamethasone can be typically used at concentrations between about 1 mM and 1 μM, preferably between about 1 nM and 100 nM, e.g., at about 10 nM. In specific GMP manufacturing conditions, EGF can be absent.

Hormones can also be used in cell culture, for example D-aldosterone, diethylstilbestrol (DES), dexamethasone, insulin, estradiol, hydrocortisone, prolactin, progesterone, hyrotropin, thyroxine, and L-thyronine. Liver cells can also benefit from culturing with triiodithyronine, α-tocopherol acetate, and glucagon. Lipids and lipid carriers can also be used to supplement cell culture media. Such lipids and carriers can include, but are not limited to cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulations.

The morphological and phenotypic features of HHALPCs described in the Examples may allow obtaining such cells not only when cryopreserved preparations of primary liver cells have low plating efficiency, but also by testing and/or adapting known technologies for preparing adherent cells from heterogeneous preparations of primary cells by selecting and combining different technologies, conditions, and/or materials (e.g. the synthetic polymeric material, the component(s) of extracellular matrix, the cell culture medium, the amount or oxygen and/or CO₂ in the incubator, the washing buffer, etc.). In particular, culturing in hypoxic conditions (as obtained by adding an anti-oxidant compound at millimolar or lower concentrations), together with one or more combinations of these other elements can be applied in order to obtain HHALPCs in greater amount and/or more quickly.

This step of culturing of primary liver cells as defined above leads to emergence and proliferation of HHALPCs in the culture and can be continued until HHALPCs have proliferated sufficiently. For example, culturing can be continued until the cell population achieved a certain degree of confluence (e.g., at least 50%, 70%, or at least 90% or more confluent). The term “confluence” as used herein refers to a density of cultured cells in which cells contact one another, covering substantially all of the surfaces available for growth (i.e., fully confluent).

Concerning Step (d) of the method, primary cells are cultured in a cell culture medium sustaining their adherence and the proliferation of and the emergence of a homogenous cell population that, following at least one passage, is progressively enriched for HHALPCs. HHALPCs can be rapidly expanded for generating sufficient cells for obtaining HHALPC Progeny having the desired properties (e.g. as bi-dimensional adherent cells or three-dimensional cell clusters, at a given density and/or differentiation status), with cell doubling that can be obtained within 48-72 hours and maintenance of HHALPC Progeny having the desired properties for at least for 2, 3, 4, 5 or more passages.

When passaged, the cultured cells are detached and dissociated from the culture substrate and from each other. Detachment and dissociation of the cells can be carried out as generally known in the art, e.g., by enzymatic treatment with proteolytic enzymes (e.g., chosen from trypsin, collagenase, e.g., type I, II, III or IV, dispase, pronase, papain, etc.), treatment with bivalent ion chelators (e.g., EDTA or EGTA) or mechanical treatment (e.g., repeated pipetting through a small bore pipette or pipette tip), or any combination of these treatments.

A suitable method of cell detachment and dispersion should ensure a desired degree of cell detachment and dispersion, while preserving a majority of cells in the culture. Preferably, the detachment and dissociation of the cultured cells would yield a substantial proportion of cells as single, viable cells (e.g., at least 50%, 70%, 90% of the cells or more). The remaining cells may be present in cell clusters, each containing a relatively small number of cells (e.g., on average, between 1 and 100 cells).

Next, the so detached and dissociated cells (typically as a cell suspension in an isotonic buffer or a medium) may be re-plated onto a substrate which allows the adherence of cells thereto, and are subsequently cultured in a medium as described above sustaining the further proliferation of HHALPCs and of HHALPC Progeny. These cells may be then cultured by re-plating them at a density of between 10 and 10⁵ cells/cm², and at a splitting ratio between about 1/16 and 1/2, preferably between about 1/8 and 1/2, more preferably between about 1/4 and 1/2. The splitting ratio denotes the fraction of the passaged cells that is seeded into an empty (typically a new) culture vessel of the same surface area as the vessel from which the cells were obtained. The type of culture vessel, as well as of surface allowing cell adherence into the culture vessel and the cell culture media, can be the same as initially used and as described above, or may be different. Preferably, cells are maintained onto CellBind or any other appropriate support that is coated with extracellular matrix proteins (such as collagens, and preferably collagen type I) or synthetic peptides that are acceptable in GMP conditions.

Concerning step (e) above, the isolation of population of HHALPCs applies to cells that are positive for the listed markers, further validating the criteria for initially identifying HHALPCs at step (c) above but that can be more easily established given the higher amount of cells that are available after passaging.

The terms “isolating” or “isolation” refers to both the physical identification and the isolation of a cell population from a cell culture or a biological sample that can be performed by applying appropriate cell biology technologies that are either based on the inspection of cell cultures and on the characterization (and physical separation when possible and desired) of cells corresponding to the criteria, or on the automated sorting of cells according to the presence/absence of antigens and/or cell size (such as by FACS). In some embodiments, the terms “isolating” or “isolation” may comprise a further step of physical separation and/or quantification of the cells, especially by carrying out flow cytometry.

The terms “cell population” and “population of cells” refer generally to a group of cells. Unless indicated otherwise, the term refers to a cell group consisting essentially of or comprising cells as defined herein. A cell population may consist essentially of cells having a common phenotype or may comprise at least a fraction of cells having a common phenotype. Cells are said to have a common phenotype when they are substantially similar or identical in one or more demonstrable characteristics, including but not limited to morphological appearance, the level of expression of particular cellular components or products (e.g., RNA or proteins), activity of certain biochemical pathways, proliferation capacity and/or kinetics, differentiation potential and/or response to differentiation signals or behaviour during in vitro cultivation (e.g., adherence or monolayer growth). Such demonstrable characteristics may therefore define a cell population or a fraction thereof. A cell population may be “substantially homogeneous” if a substantial majority of cells have a common phenotype. A “substantially homogeneous” cell population may comprise at least 60%, e.g., at least 70%, at least 80%, at least 90%, at least 95%, or even at least 99% of cells having a common phenotype, such as the phenotype specifically referred to HHALPCs (or to HHALPC Progeny). Moreover, a cell population may consist essentially of cells having a common phenotype such as the phenotype of HHALPCs (i.e. an HHALPC Progeny) if any other cells present in the population do not alter or have a material effect on the overall properties of the cell population and therefore it can be defined as a cell line.

In general, any technology for identifying and characterizing cellular markers for a specific cell type (e.g. mesenchymal, hepatic, hematopoietic, epithelial, endothelial markers) or having a specific localization (e.g. intracellular, on cell surface, or secreted) that are published in the literature may be considered appropriate for characterizing HHALPCs and HHALPC Progeny. Such technologies may be grouped in two categories: those that allow maintaining cell integrity during the analysis, and those based on extracts (comprising proteins, nucleic acids, membranes, etc.) that are generated using such cells. The Examples contain data on how such technologies have been used for characterizing HHALPCs and HHALPC Progeny, e.g. by performing an analysis of the presence of cell surface antigens before performing a more detailed and comparative analysis with other liver progenitor cells or adult liver primary cells in order to assess their distinctive features and biological activities.

At the protein level, technologies such as flow cytometry or immunocytochemistry, allow determining the presence/absence of surface or intracellular proteins in HHALPCs by using antibodies or other protein-specific reagents. Flow cytometry is a preferred technology for characterizing cell populations according to the combined presence/absence of surface, or intracellular markers, as determined by single or multiple staining techniques, and/or size and granularity evaluation. Immunocytochemistry also provides information regarding morphological features that are associated to the combined presence/absence of surface, cytoskeletal, and/or other intracellular markers.

In particular, the presence of at least one mesenchymal marker, one adhesion marker, one tetraspanin, one marker selected from CD98, CD140b, and β2-microglobulin, and of at least one hepatic marker should be measured by flow cytometry, immunocytochemistry, or any other technique (generally making use of antibodies, lectins, or other proteins and not requiring the protein or nucleic acid extraction) that allows evaluating the percentage of cells presenting the receptor. The positivity for additional cell surface markers other than those strictly associated to hepatic or mesenchymal features (such as the positive marker that are mentioned in the Examples) can be similarly measured. Positivity by flow cytometry and immunocytochemistry is here defined when at least 60% of cells present the desired marker or receptor (as shown in the Examples). Similarly, the negativity by flow cytometry and immunocytochemistry is here defined when less than 20% of cells present the given marker or receptor (as shown in the Examples). In some embodiments, less than 10% of cells present a given negative marker. When referring to cell surface markers, the positivity is preferably measured in non-permeabilized cells.

In some embodiments, when measuring a given marker, the agent that is used for detection of a marker as defined above or a cell surface protein is immobilized on a solid phase (e.g. a bead, a plate, or a biomaterial), labelled (e.g. fluorescently labelled), and/or recognized by another compound that is labelled (e.g. a secondary antibody). There are numerous methods by which the label can produce a signal detectable by external means, for example, desirably by visual examination or by electromagnetic radiation, heat, and chemical reagents. The label or other signal producing system component can also be bound to a specific binding partner, another molecule or to a support such as beads, using any method known in the art, such as chemically cross-linking or using the biotin-streptavidin system. The label can directly produce a signal, and therefore, additional components are not required to produce a signal. Numerous organic molecules, for example fluorochromes (such as FITC, PE, PC5, PC7, APC, or any other known to be compatible with flow cytometry), absorb ultraviolet and visible light. Other types of label directly produce a signal, such as radioactive isotopes and dyes. Alternatively, the label may need other components to produce a signal, and the signal producing system would then include all the components required to produce a measurable signal, which may include substrates, coenzymes, metal ions, or substances that react with enzymatic products (e.g. chemiluminescent detection of Horseradish Peroxidase).

The liver-specific metabolic activities of HHALPCs comprise biological activities generally associated with liver cells (and to hepatocytes in particular) and that distinguish liver cells from cells present in other tissues, and in particular comprise activities involving binding, activation, and/or degradation of proteins or other substrates as described in the literature and in the Examples. These biological activities are established on the basis of the detection of liver-specific metabolic activities that can be protein/drug binding activities and, more preferably, enzymatic activities on given substrates, or in association to liver-specific molecules that are detected by blotting technologies (Western, or Northern blot), sequencing, isoelectrofocusing, ELISA, or of the internalization of synthetic or natural compounds known to be specifically transported and metabolized within liver cells. Other relevant enzymatic activities other than those strictly associated to hepatic features can be similarly measured and compared with those measured within hepatocytes or other cell types using techniques that are described in the literature. Depending on alternative approaches and uses, activities related to endothelium (e.g. in connection to the passage across this barrier and reach tissues) or blood (e.g. in connection to coagulation) can be measured in vitro or in appropriate in vivo models.

At the nucleic acid level, whole genome sequencing, PCR, or RT-qPCR can be used to characterize HHALPCs or HHALPC Progeny. Hereby, real time PCR can be used to quantify the expression of the gene under investigation, based on the number of cycles and having it normalized against the cycles obtained for 1 or more endogenous controls. In particular, the RT-PCR reaction can be performed using HHALPCs and appropriate primers and buffers but the number of cycles to obtain a signal should not be superior to 25, 30 or 35 cycles.

At the activity level, the presence of a liver-specific metabolic activity can be measured by any appropriate technique that allows evaluating the presence and/or the level of activity of liver-specific enzymes, but preferably should allow quantifying in vitro the actual enzymatic activity, with a given limit of detection of the specific end-product (as it can be easily established with the support of literature and commercially available products) for measuring CYP450 activities, detoxification, glycogen storage, secretion of Alpha-1-Antitrypsin or albumin, bile production, thrombopoietin production, angiotensinogen production, conversion of ammonia to urea, cholesterol synthesis, glycogenolysis, glycogenesis and lipogenesis. In particular, the positivity for at least a liver-specific metabolic activity is here defined when the activity is measured as being statistically superior to the limit of detection of the end-product (being at least twice, five times, or ten times more than the limit of detection) or approaching the level of activity of primary hepatocytes (superior, identical or at most 10%; at most 25%, at most 50%, at most 75%, or at most 90% lower).

The literature provides extensive description of the technologies for evaluating cytochrome P450 activities in human hepatocytes in vitro, in particular regarding the compounds specifically inducing an enzyme activity and the formats that can be used for performing these experiments (Gerets H H et al., 2012; Gomez-Lechon M J et al., 2012). Among the different inducers, drug metabolism in these cells can be assessed using midazolam, ethoxyresorufin, benzoxyresorufin, bupropion, Phenacetin, Diclofenac, tolbutamide, phenobarbital, rifampicin, caffeine, beta-naphthoflavone, omeprazole, dextromethorphan, 3-methylcholanthrene, repaglinide, or other known cyto/hepatotoxic compounds as probes that are listed in the literature (Bale S et al., 2014). Metabolite detection and quantification can be associated to the activity of hepatic enzymes on specific compounds such as CYP1A2 (by detecting paraxanthine or acetaminophen), CYP3A4 (by detecting 1-OH-midazolam or omeprazole sulfone), CYP2C6 (by detecting HO-Bupropion), CYP2C9 (by detecting 4′HO-Diclofenac), as well as for other major cytochrome P450 activities such as CYP1A2, CYP3A5, CYP3A7, or CYP7A1 (singularly or in appropriate combinations).

Other enzymes whose expression or (preferably) activity can be established in HHALPCs and HHALPC Progeny are UDP-glucuronosyltransferases (such as UGT1A1, UGT2B4, UGT2B7), sulfotransferases (catalyzing the sulfate conjugation of several pharmacologically important endogenous molecules and xenobiotics), tyrosine transferases, tryptophan-2,3-dioxygenase (TDO2 or TDO), indoleamine-2,3-dioxygenases (IDO1 or IDO2), lysyl oxidase (LOX), glutathione S-transferases (e.g. GSTalpha), multidrug resistance proteins (MDR or MRP-1/-2/-3), liver-specific transporters (such as OATP1B1), and other phase I/II/III biotransformation enzymes. Moreover, albumin/urea production and secretion, ammonia metabolism, glycogen storage, bile production, thrombopoietin/angiotensinogen production, and galactose/sorbitol elimination rates can be also measured and compared by applying well-established protocols.

When a preparation of HHALPCs is obtained by the methods of the invention, this cell population can be maintained and/or propagated in conditions that allow growth and doubling without differentiation. Preferably, HHALPCs are passaged, as non-differentiated adherent cells, no more than 2, no more than 3, no more than 4, or o more than 5 times in culture, so that the number of cell doublings can be evaluated for establishing the most appropriate conditions for further in vivo or in vitro use. After one or more passaging in this status, HHALPCs can be induced to differentiate into hepatocyte-like or hepato-active cells. In both cases, the resulting cells represent HHALPC Progeny. In the first case, the conditions for maintaining HHALPCs as undifferentiated HHALPC Progeny may be the same ones used for obtaining the original population of HHALPCs with the purpose of increasing the number of available cells.

Following step (e) of the methods of the invention, an optional further step (f) may comprise maintaining HHALPCs into cell culture conditions allowing the differentiation into cells presenting liver-specific activities, being for instance hepatocyte-like or hepato-active cells (that is, adult liver progenitor cells that have lost their positivity to most, if not all, mesenchymal markers and are positive for most, if not all, morphological, biological and functional features of hepatocytes). Alternatively, the HHALPC Progeny present a combination of hepatic and other tissue-specific features that relate to the specific GMP manufacturing process, formulation, site of administration, or concurrent use of other compounds in vivo. These properties may be related in particular to the proteins having immunoregulatory features that are expressed on the cell surface or secreted, as described in the literature for ADHLSCs (Berardis S et al., 2014; Sana G et al., 2014; Raicevic G et al., 2015).

The additional passages (e.g., cell detachment and dispersion, re-plating, etc.) and culturing (e.g., medium addition or changes following confluence, etc.) may be performed at conditions substantially identical or analogous to those of the first passage, as described above or including modifications which would be suggested in the literature and/or for the specific use of HHALPCs or HHALPC Progeny. Thus, the conditions for maintaining and/or differentiating HHALPCs or HHALPC Progeny in cell culture may be further optimized according to different criteria such as timing/medium for the differentiation into hepatocyte-like or hepato-active cells, systems for maintaining three-dimensional cell cultures as cell suspensions, use of specific substrates or scaffolds, hypoxia, combined or sequential addition of growth factors and chemical compounds within cell culture medium, or cell density.

During such later passages, the activity and overall phenotype of HHALPC Progeny may be further adapted and/or improved for final storage, formulation, and/or use function by engineering the cells at the level of exposed cell surface proteins and/or their glycosylation in vitro, without genetic manipulation, in particular for improving cell engraftment for example by adding Sialyl lewis X groups or peptides (Sarkar D et al., 2011; Wan X et al., 2013; Cheng H et al., 2012). HHALPC Progeny may be also cultured in specific conditions at final stage (just before use or storage) so that some properties are better maintained for example by culturing on a thermosensitive polymer or other supports that allow a milder release of cells (Nash M et al., 2013; You J et al., 2013; Nagase K et al., 2015). Further modifications of the cell culture medium (such as culturing in hypoxic conditions with antioxidants, or by adding cytokines or other compounds such as lycopene) that may improve cell engraftment, in vivo activities, or reduce apoptosis, can be applied as a pre-treatment in accordance to the literature (Kavanagh D et al., 2014; Kim J Y et al., 2015; Zeng W et al., 2015).

The methods of the Invention provide HHALPCs, presenting morphological, protein expression, and functional features that are distinct from those identified in previously described adult progenitor liver cells. Consequently, HHALPCs that are obtained or obtainable by the methods defined above represent a further embodiment of the Invention. These methods allow providing cell populations comprising a high proportion of the specific cells (at least 30%, 40%, 50%, 60%, 70%, 80%, 90% or more), even yielding a substantially homogeneous cell population as it can be evaluated by any appropriate standard method, e.g., by flow cytometry or any other immunostaining approach, with or without evaluating further biological activities.

HHALPCs and HHALPC Progeny can be used for establishing cell cultures for any immediate use or stored as cryopreserved preparations each containing at least 10³, 10⁶, 10⁹ cells or more, aimed to produce or use higher amount of HHALPCs or HHALPC Progeny after appropriately thawing the preparations and, if needed, for producing HHALPCs and HHALPC Progeny a the industrial scale (e.g. using bioreactors, membranes, microspheres, microfluidics, or any other technical solution for improving bioprocessing and cell expansion while maintaining desired cell properties). Samples of cell populations corresponding to any of the HHALPCs and HHALPC Progeny may be cryostored in a serum-containing or serum-free preservation medium (e.g. commercially available cryopreservation formulations) and/or in the presence of a cryoprotecting agent (e.g. dimethyl sulfoxide at an appropriate concentration). HHALPC Progeny can be compatible with commercial systems that are developed for organ-on-a-chip applications in safety testing, pathophysiological studies, and other cell-based microfluidic liver models for drug development and toxicology (Alépée N et al., 2014; Lin C et al., 2015).

In particular, preparations of HHALPCs and HHALPC Progeny comprising a predetermined number of cells (e.g. 50000, 100000, 500000, 1 million, 10 million, 100 million, 1 billion or more cells) can be provided in one or more vials that can be then included in a kit comprising such vials (or other appropriate vessel or container such as those used for microfluidics applications) that can be then included in a kit comprising such vials, containers, microfluidics apparatus, and any other appropriate device, disposable materials (e.g. filters, syringes), solutions (e.g. PBS, cell culture medium, diluent), chemicals (e.g. enzymatic substrates, fluorochromes, drugs), biological products (e.g. growth factors, antibodies, primers) and/or instructions for using the components of such kit that can be appropriately packaged and sent to clients for using HHALPCs and HHALPC Progeny in vivo (e.g. for the administration to a patient or to animal) or in vitro (e.g. for testing toxicity or efficacy of compounds as candidate drugs) consequently.

The maintenance, proliferation, and/or differentiation of HHALPCs and HHALPC Progeny in cell culture conditions (or following implantation in an animal model or in a patient) can be performed as required for the desired use. The literature provides several protocols for maintaining liver progenitor cells and/or generating from them hepatocyte-like or hepato-active cells. The Examples provide means for obtaining HHALPCs and HHALPC Progeny in cell culture conditions, and for differentiating them into cells presenting liver-specific activities in the form of adherent cells or as three-dimensional cell clusters.

In this latter case, HHALPCs and HHALPC Progeny can be provided for the desired use as three-dimensional cell clusters similar to the liver spheroids or organoids that, according to the literature, may provide cells with significant improvements in viability and functionality when administered intra- or extrahepatically, used for testing the hepatotoxicity of compounds, maintained as cryopreserved preparations, expanded in bioreactors or multi-tray stacks for upscaling manufacturing process, or used in liver assist devices (Ebrahimkhani M et al., 2014; Lancaster M A et al., 2014; Massie I et al., 2011). HHALPCs and HHALPC Progeny may be obtained also by encapsulating the cells in synthetic or biological matrices. In particular, liver decellularized scaffolds or extracellular matrices may be used as scaffolds for culturing one or more cell types, as two-dimensional substrate coating and three-dimensional injectable hydrogel platform for generating hepatic organoids (Lee J et al. 2014; Caralt M et al., 2014).

The maintenance, proliferation, and/or differentiation of HHALPCs and HHALPC Progeny can be improved by adapting cell culture conditions using technical solutions well known in the art for stem, progenitor, or mesenchymal cells of different origin. For example, ex-vivo protocols of non-cell damaging low oxygen atmosphere and other approaches for adapting in vitro microenvironments may facilitate survival, genetic stability, proliferation, post-engraftment differentiation, homing and repopulation in the liver, secretion of paracrine factors, and overall therapeutic potential of such cells (Muscari C et al., 2013; Cigognini D et al., 2013). Otherwise, human blood-derived components such as umbilical cord blood serum and platelet lysate are tested and developed as cell culture components that are non-xenogenic alternative to bovine serum and still compliant with GMP guidelines to yield clinically cell compositions, without the well-known problems associated to serum such variability in the quality, risk of contamination, and undesired immunizing effects (Bieback K, 2013).

Before being administered or otherwise used, HHALPCs and HHALPC Progeny can be transiently or stably modified by exposing said cells to heterologous biological or chemical agents, or by introducing said agents into the cells. In particular HHALPCs and HHALPC Progeny can be modified (or engineered, following their transformation with appropriate vectors) in cell culture (e.g. after and/or before their differentiation) by treating cells with growth factors and/or introducing nucleic acids that affect overall expression profile of the cells, preferably towards specific hepatic features or features helping cell culture (e.g. by transducing cells with microRNAs or with lentiviral vectors expressing recombinant proteins, such as growth factors, or transcription factors known to affect hepatic differentiation or the differentiation towards any other cell type, and/or the production of proteins of therapeutic interest, or fluorescent proteins).

In particular, HHALPCs and HHALPC Progeny may consequently present improved and/or additional biological activities in vivo and/or in vitro, after and/or before their differentiation into cells presenting a full range of liver-specific activities. Preferably, HHALPCs and HHALPC Progeny are engineered before being differentiated so that HHALPC Progeny is consistently modified to have improved biological activities, independently from any later in vitro differentiation or in vivo use (that may imply, or not, hepatic or other type of differentiation).

The treatment of HHALPC Progeny with chemical agents, cell culture medium, and/or nucleic acid vectors that are known as inducing the differentiation of other known liver progenitor/stem cells into other non-hepatic cell types (e.g. osteocytes, insulin-producing beta cells, or bone marrow cells) may equally provide such non-hepatic cell types. Non-hepatic cell populations that are obtained by applying these technologies known in the literature to HHALPCs (or any specific type of HHALPC Progeny) are additional types of differentiated HHALPC Progeny than the one described in the Examples (obtained by using a cell culture medium for inducing hepatic differentiation) that can be used in vitro and/or in vivo (in particular for therapeutic uses) according to the biological activities that the HHALPC Progeny has lost and/or acquired as a consequence to such treatment (e.g. a differentiated HHALPC Progeny that produce and secrete insulin may be used for treating diabetes).

Conventional gene transfer methods applicable to liver progenitor cells can be used to introduce nucleic acids into HHALPCs and HHALPC Progeny, including microinjection, electroporation, co-precipitation with calcium phosphate, liposomes, or viral transfection. Following their transformation with appropriate vectors, HHALPCs and HHALPC Progeny may express recombinant proteins or contain nucleic acids that allow said cells performing improved and/or additional biological activities in vivo and/or in vitro, after and/or before their differentiation into hepatocyte-like or hepato-active cells (for instance, at scope of establishing liver progenitor cell-based models for gene therapy). When the vectors are viral vectors (e.g., a lentivirus vector), they will be characterized by determination of their titre in order to select the optimal transduction efficiency conditions and proliferation rate, and to analyse their expression profile as well as their safety.

The liver is anatomically connected with the circulatory system in such a way that it allows an efficient release of various proteins into the bloodstream. Therefore, genes encoding proteins that have systemic effects may be inserted into HHALPCs and HHALPC Progeny (in particular before being cultured for obtaining three-dimensional cell clusters) for further improving their efficacy (in particular when administered systemically, e.g. by intravenous, intramuscular, or intraperitoneal injection), as well as for their engraftment and maintenance when administered in vivo.

For example, a variety of genes coding for hormones or antibodies may be inserted into liver cells of the present invention for the secretion of their gene products into the circulation. In particular, HHALPCs and HHALPC Progeny may be modified to constitutively or transiently over-express a protein normally expressed by hepatocytes (and possibly already expressed by such cells), but being defective or absent in a patient (this defect underlying a pathological state of the patient, as in inborn errors of liver metabolism) and then helping restoring production of the protein and thereby helping in the treatment of the patient. Examples of such proteins are metabolic enzymes such as lyase, arginase, glucokinase, ornithine transcarbamylase, arginosuccinate synthetase, arigininosuccinate carbamyl phosphate synthase, N-acetyl glutamate synthase, glutamine synthetase, glycogen synthetase, glucose-6-phosphatase, alkaline phosphatase, succinate dehydrogenase, pyruvate kinase, acetyl CoA carboxylase, fatty acid synthetase, alanine aminotransferase, glutamate dehydrogenase, Cytochrome P450 enzymes, aldehyde dehydrogenases, and/or alcohol dehydrogenase. Alternatively, HHALPCs and HHALPC Progeny may be modified by introducing the DNA encoding a secreted plasma protein such as albumin, a growth factor or hormone, insulin, transferrin, complement proteins (such as component C3), alpha2-macroglobulin, fibrinogen alpha/beta/gamma chain, coagulation Factors (Factor V, Factor VII, Factor VIII, Factor XI, Factor XIII, Factor IX), alpha-1-antitrypsin, or the like.

Biological materials that are obtained when generating HHALPCs and HHALPC Progeny can be further used for identifying biological entities that may have specific uses, in particular distinct medical applications. These biological materials include not only sub-population (or cell lines) of HHALPCs or of HHALPC Progeny that present specific markers, activities, and/or morphology (as determined in Examples 3 and 4) but also any other biological entity that is obtained as intermediate or final products, such as conditioned cell culture media and fractions of these cells and media including proteins, metabolites, cell vesicles, and/or nucleic acids that can be used as biomarkers for detecting cells of medical interest or as compounds that present activities or distribution of medical interest. Additional information can be also determined by measuring the content of the conditioned cell culture media (e.g. in the form of a cell culture supernatant) which can provide relevant information on the secretome and in particular on the paracrine effects of HHALPCs and of HHALPC Progeny.

Relevant biological features of HHALPCs or HHALPC Progeny can be identified by using technologies such as flow cytometry, immunocytochemistry, mass spectrometry, gel electrophoresis, an immunoassay (e.g. immunoblot, Western blot, immunoprecipitation, ELISA), nucleic acid amplification, enzymatic activity, omics technologies (proteomics, glycomics, transcriptomics, metabolomics) and/or other biological activity. In particular, 'omics technologies may provide additional means for comparing HHALPCs or HHALPC Progeny using databases and other datasets that are published for stem or progenitor cells, and in particular for liver progenitor cells (Yu J, et al., 2012; Santamaria E, et al., 2012; Slany A, et al., 2010; Sison-Young R et al., 2015). These additional markers can be used either during the initial step of HHALPC Progeny preparation or later on (e.g. for comparing and validating industrially manufactured batches of HHALPC Progeny or for evaluating suitability for pharmaceutical use).

These approaches may provide means for defining novel biomarkers associated to adult liver progenitor cells, either in vivo or in vitro (e.g. for establishing quantity, quality and homogeneity of a cell population before, during, or after its preparation and use). In particular, the biomarkers can be defined by means of the concentration of a given cell population (HHALPCs and/or HHALPC Progeny) in a biological sample or in a cell culture in general or in combination with the concentration of cells that present a specific protein, lipid, enzyme, phospholipid, and/or glycan. Such biomarkers can correspond to a peptide, a protein, a phospholipid, a lipid, a nucleic acid, a glycan, or any combinations of such elements components. The biomarker can be specific for assessing the suitability of a cell population being HHALPCs or a HHALPC Progeny, for a given use (e.g. treating a specific liver disease, obtaining hepato-active cells types following in vitro differentiation or modification with chemical agents and/or nucleic acid vectors, assessing the metabolism of a specific compound), in particular when comparing HHALPC Progeny obtained from different donors and/or by applying different manufacturing processes. Otherwise, the biomarker allows assessing if a given liver tissue (or sample of fresh or cryopreserved liver cells) is appropriate for obtaining HHALPCs more efficiently (e.g. by screening banks of liver tissues and libraries of other liver-originated biological samples such as protein extracts and cDNA libraries) for establishing which donors and/or samples can be selected).

The term “biomarker” or “marker” refers to a molecule, a parameter, a characteristic, or an entity that is objectively measured and evaluated as characterizing HHALPCs and or HHALPC Progeny. The quantitative evaluation of a biomarker that is associated to HHALPCs and/or HHALPC Progeny in a specific sample (such a tissue or a biological fluid) can be associated to a quantitative evaluation of total cells, to the efficiency with which HHALPCs and/or HHALPC Progeny can be produced and isolated, to a specific in vitro technology, or to a specific medical use or status of a patient.

HHALPCs and HHALPC Progeny can be used in regenerative medicine and in biological assays requiring cells that present biological features (such as metabolic or enzymatic activities, an antigenic profile, or other phenotype) as similar as possible to those observed for primary hepatocytes for the desired period of time, once they are differentiated either in vivo or in vitro, or even before inducing a full differentiation towards cells presenting a larger number and/or stronger liver-specific activities (that is, hepato-active cells). HHALPCs and HHALPC Progeny can be also used for in vitro applications such as pharmacological or toxicological studies (e. g. screening and characterization of biological or chemical agents) HHALPCs and HHALPC Progeny allow establishing of in vitro and animal models of toxicology, pharmacology and pharmacogenetics (as extensively described for primary hepatocytes and hepatocyte-like cells derived from progenitor or stem cell of various origin) or identification of biomarkers for identifying in vivo and/or in vitro cell population of medical interest, in particular in connection to the diagnosis, the prevention, and/or the treatment of liver diseases.

The term “in vitro” as used herein denotes outside, or external to, animal or human body. The term “in vitro” as used herein should be understood to include “ex vivo”. The term “ex vivo” typically refers to tissues or cells removed from an animal or human body and maintained or propagated outside the body, e.g., in a culture vessel or a bioreactor.

If HHALPCs and HHALPC Progeny may be preferably used for in vivo applications, the HHALPC Progeny that are differentiated in vitro may be preferably used as differentiated hepatocyte-like or hepato-active cells for drug discovery/validation

HHALPCs and HHALPC Progeny (or corresponding biological materials that are obtained when generating them) can be provided in compositions comprising them, and in particular as pharmaceutical compositions that can be used in therapeutic methods for in vivo administration (in humans or in animal models) or in vitro applications in the form of a composition including such cells either as fresh cells or cells suitable for long-term storage (e.g. cryopreserved cells).

Preferably, a composition comprising HHALPCs or HHALPC Progeny may comprise at least 10³, 10⁶, 10⁹ or more cells (for example, between 5 million and 500 million or between 5 million and 250 million or between 50 million and 500 million or between 50 million and 250 million or between 100 million and 500 million or between 100 million and 250 million of cells for each dose or administration). Such cell-based compositions may also include other agents of biological (e.g. antibodies or growth factor) or chemical origin (e.g. drugs, cell preserving or labelling compounds) that may provide a further therapeutic, diagnostic, or any other useful effect. The literature provides several examples of optional additives, excipients, vehicles, and/or carrier that are compatible with cell-based pharmaceutical compositions that may include further specific buffers, growth factors, or adjuvants, wherein the amount of each component of the composition is defined (in terms of micrograms/milligrams, volume, or percentage), as well as the means to combine them with HHALPCs and HHALPC Progeny.

HHALPCs and HHALPC Progeny can be administered in the form of a composition which depending on chosen administration method, can be a suspension of cells, a sponge or other three-dimensional structure where cells can grow and differentiate in vitro and/or in vivo including bioartificial liver devices, natural or synthetic matrices, or other systems allowing the engraftment and functionality of cells in appropriate locations (including areas of inflammation or tissue injury that expressing chemokines that help the homing and the engraftment of the cells). In particular, HHALPCs and HHALPC Progeny can be administered via injection (encompassing also catheter administration, intravenously or intra-arterially) or implantation, e.g. localised injection, systemic injection, intrasplenic injection, intra-articular injection, intraperitoneal injection, intraportal injection, injection to liver pulp, e.g., beneath the liver capsule, parenteral administration, or intrauterine injection into an embryo or foetus.

When systemically and not locally injected, the HHALPC Product may have an effect in a distant location either because such HHALPCs move in the bloodstream and engraft in distant locations (such internal organs or joints), or the proteins secreted by HHALPCs reach specific cell types, thanks to bloodstream. Moreover, HHALPCs and HHALPC Progeny can be used biological components of detoxification devices such as liver perfusion or liver assist devices with rigid, plastic outer shell and hollow semi-permeable membrane fibres in which HHALPCs or HHALPC Progeny (like other stem cells, primary human cells such as differentiated hepatocytes, or cell types derived from stem cells) are seeded. Bodily fluid can be perfused through the device for detoxification according to well-known procedures and then returned to the patient.

HHALPCs, HHALPC Progeny or composition containing them can be used for tissue engineering and cell therapy via liver cell transplantation (LCT) in intrahepatic or extra-hepatic locations (including for modulating immunological response to the prior or subsequent transplantation of liver or other organs and tissues). Using this approach, animal models of human liver diseases can be also obtained by transplanting HHALPCs of human origin, HHALPC Progeny of human origin, or a composition containing them in animals wherein the effects of a compound on human hepatocytes can be more effectively evaluated and distinguished from effects in the animal model.

When administering a therapeutic composition comprising HHALPCs or a specific HHALPC Progeny, it may generally be formulated in a unit dosage. In any case, it may be desirable to include agents and/or adapt known methods for administering cells to patients that ensure viability of HHALPCs or HHALPC Progeny, for example by incorporating the cells into a biopolymer or synthetic polymer. Examples of suitable biopolymers include, but are not limited to, fibronectin, fibrin, fibrinogen, thrombin, collagen, and proteoglycans laminins, adhesion molecules, proteoglycans, hyaluronans, glycosaminoglycan chains, chitosan, alginate, natural or synthetically modified peptides that are derived from such proteins, and synthetic, biodegradable and biocompatible polymers. These compositions may be produced with or without including cytokines, growth factors, and administered as a suspension or as a three-dimensional gel with the cells embedded there within.

The methods of the invention contemplate not only using any donor of liver tissues for generating HHALPCs or HHALPC Progeny but using a patient's own liver tissue to produce and isolate HHALPCs and generating HHALPC Progeny or composition containing them. Such cells would be autologous to the patient and could be readily administered to the patient. Otherwise HHALPCs may be produced and isolated from tissue which is not patient's own. Where administration of such cells to a patient is contemplated, it may be preferable that the liver tissue subjected to the method of the present invention to obtain HHALPCs is selected such as to maximize, at least within achievable limits, the tissue compatibility between the patient and the administered cells, thereby reducing the chance of rejection of the administered cells by patient's immune system (e.g., graft vs. host rejection).

An issue concerning the therapeutic use of HHALPCs and HHALPC Progeny is the quantity of cells necessary to achieve an optimal effect. Doses for administration may be variable, may include an initial administration followed by subsequent administrations; and can be ascertained by the skilled artisan by applying the teaching of the present disclosure. Typically, the administered dose or doses will provide for a therapeutically effective amount of the cells and it may require optimization of the amount of administered cells. Thus, the quantity of cells to be administered will vary for the subject being treated (e.g. between 10² to 10¹⁰ cells per each treatment in a cycle or for the entire cycle of treatment; e.g., between 1×10⁶ and 1×10⁷ cells/Kg body weight or between 2×10⁶ and 8×10⁶ cells/Kg body weight or between 3×10⁶ and 5×10⁶ cells/Kg body weight of the subject, per each treatment in a cycle; or, e.g., between 1×10⁶ and 1×10⁸ cells/Kg body weight or between 5×10⁶ and 5×10⁷ cells/Kg body weight or between 1×10⁷ and 2×10⁷ cells/Kg body weight of the subject, for the entire cycle of treatment). However, the precise determination of a therapeutically effective dose may be based on factors individual to each patient, including their size, age, size tissue damage, and amount of time since the damage occurred.

Preferably, compositions comprising HHALPCs or a specific HHALPC Progeny should contain a substantially homogeneous cell population as defined above and the amount of cells within each dose can be consequently adjusted.

The distribution, differentiation, and/or proliferation of HHALPCs or HHALPC Progeny after their administration or implantation can be determined (as well as their activity after/before the administration of a different therapeutic agent) can be tested in human subject or in animal models (preferably a rodent). For example, the analysis of the livers of SCID mice intrasplenically transplanted with HHALPCs or HHALPC Progeny may demonstrate that these cells are able to engraft in the liver and repopulate the organ by detection of a human marker, and to differentiate into active, mature hepatocytes by detection of human albumin, or any other typical human liver-specific marker (or a recombinant gene that was previously transfected in the administered HHALPCs or HHALPC Progeny).

Another aspect of the invention is a method for preventing and/or treating a liver disease, comprising administration of HHALPCs, HHALPC Progeny or a composition containing them to a subject in need thereof. HHALPCs and HHALPC Progeny can be used for treating liver diseases, in particular those requiring the permanent (or time-limited) re-establishment of liver function in a subject that, according to the literature, requires liver transplantation, hepatocyte transplantation, or liver regeneration given the loss of liver mass and/or function that is observed and that can be grouped in different categories.

A method for treating a liver disease comprises administering an HHALPC Product, such as HHALPCs or a given HHALPC Progeny, and preferably within a composition, to a subject in need thereof. In particular, a method of treating a disease in a patient in need thereof comprises administering an effective amount of an HHALPC Product to the patient, the disease being preferably a liver disease or an inherited Blood Coagulation Disorder.

A first category of liver diseases is represented by inborn errors of liver metabolism that can be further distinguished into errors of amino acid metabolism (such as Maple Syrup Urine Disease, Phenylketonurias, Tyrosinemia, Propionic Acidemia, Organic Aciduria, and Urea Cycle Disorders including Argininosuccinic Aciduria, Carbamoyl-Phosphate Synthase I Deficiency, Citrullinemia, Hyperargininemia, and Ornithine Carbamoyltransferase Deficiency), of metal metabolism (such as Wilson's Disease or Hemochromatosis), and of carbohydrate metabolism (such as Glycogen Storage Disease type I/II, fructosemia, or Galactosemias), lysosomal disorders (such as Wolman disease, Niemann Pick disease), peroxisomal disorders (such as Refsum Disease), Familial Hypercholesterolemias and other lipid metabolism disorders, mitochondrial diseases (such as Pyruvate Carboxylase Deficiency), and Hyperbilirubinemia (such as Crigler-Najjar Syndrome, Gilbert Syndrome, or Dubin-Johnson syndrome). A second category is represented by other liver diseases not directly associated to deficiencies of coagulation or metabolism and includes progressive familial intrahepatic cholestasis type 1/2/3, alpha-1-Antitrypsin Deficiency, Caroli Disease, defects of liver cell transporters, Porphyrias (such as Acute Intermittent Porphyria), fatty liver and other fibrotic liver diseases (NASH/NAFLD), primary biliary cirrhosis, sclerosing cholangitis, liver degenerative diseases, or acute or chronic liver failure (e.g. post-hepatectomy, fulminant, virally induced, acute-on-chronic liver failure), or other types of liver degeneration that may be treated by replacing liver tissues with liver progenitor cells (e. g. in liver cancer or other malignancies).

With respect to inherited blood coagulation disorders, the disease can selected such as Factor V Deficiency, Factor VII Deficiency, Factor VIII Deficiency, Factor IX Deficiency, Factor XI Deficiency, Factor XIII deficiency and other deficiencies due to the insufficient amount of other coagulation-related factors or other proteins specifically expressed and secreted by liver into blood stream, such as albumin or Tissue Factor, this latter product being expressed by such cells (Stephenne X et al., 2012) and of potential interest for some types of syndromes associated blood coagulation disorders.

As discussed above, an HHALPC Product may be administered or used in combination with another product (such as a drug, therapeutic agent, another cell type, or other biological material). This applies to any of the administrations and therapeutic uses described herein. In particular, the other therapeutic product may be administered substantially at the same time with an HHALPC Product (within the same pharmaceutical composition or in distinct pharmaceutical composition) or at different times (in distinct pharmaceutical compositions and in any order or frequency). Whether the other therapeutic product is administered separately or not from an HHALPC Product, the resulting effects may be synergic, that is the effects are superior to the additive ones that are expected, the negative effects of one of such component are mitigated or disappear, and/or the positive effects of one of such component are obtained by administering it at a lower amount or less frequently.

When the HHALPC Product is an HHALPC Progeny, these cells may be administered (being or not previously co-cultured in in vitro conditions) in combination with another cell type (e.g., primary human hepatocytes, ADHLSC Cells, or another human cell type or population being a primary, stem, mesenchymal, and/or progenitor cell such as the ones described in WO2006126219 and other progenitor or stem cells of hepatic origin) or its corresponding conditioned cell culture media, in form of a cell culture supernatant. The combination of HHALPCs with another cell type may improve the therapeutic efficacy, engraftment, homing, repopulation, proliferation, and/or stability of one and/or the other cell type within the human body. The HHALPC Progeny may be administered as part of a formulation also comprising such another cell type, or may be administered separately, but in combination with, that other cell type, such as sequentially or simultaneously (in any order). The two cell types may be administered as a suspension or co-culture of cells, or within a sponge or other three-dimensional structure where cells can grow, proliferate, and differentiate in vitro and/or in vivo including bioartificial liver devices, and natural or synthetic matrices that sustain the maintenance of these cells in the human body. The combination of HHALPCs with the conditioned cell culture media of another cell type may provide an HHALPC Progeny with improved therapeutic efficacy, engraftment, proliferation, and/or stability within the human body, together or not with further useful properties related to the composition of the conditioned cell culture media of the other cell type.

Alternatively, when the HHALPC Product is a conditioned cell culture media of an HHALPC Progeny, this preparation may be administered in combination with another cell type (e.g., primary human hepatocytes, ADHLSC Cells, or another human cell type or population being a primary, stem, mesenchymal, and/or progenitor cell such as the ones described in WO2006126219 and other progenitor or stem cells of hepatic origin) or its corresponding conditioned cell culture media. The combination of conditioned cell culture media of an HHALPC Progeny with another cell type may improve the engraftment, stability, homing, proliferation, repopulation, and/or stability of this latter cell type within the human body. Again, the HHALPC Product may be administered as part of a formulation also comprising such another cell type, or may be administered separately, but in combination with, that other cell type, such as sequentially or simultaneously (in any order). Still alternatively, the combination of conditioned cell culture media of an HHALPC Progeny with the conditioned cell culture media of another cell type may provide a solution with improved therapeutic efficacy, potentially combining the effects of the secreted proteins that are contained herein. Again, the conditioned stem cell culture media of the HHALPC Progeny may be administered as part of a formulation also comprising such another conditioned cell culture media, or may be administered separately, but in combination with, that other media, such as sequentially or simultaneously (in any order).

The administration or the therapeutic use of an HHALPC Product (similarly to the administration and the therapeutic use of ADHLSC Cells or the conditioned cell culture media of ADHLSC Cells, as described in WO2015001124) may also provide unexpected positive effects. In particular, the administration or the therapeutic use of an HHALPC Progeny or of the conditioned cell culture media obtained from an HHALPC Progeny may be combined with the administration of a specifically required protein for treating an inherited disease, such as a metabolic enzyme (e.g. Ornithine transcarbamylase or UDP-glucuronosyl transferase 1A1) or a coagulation factor (e.g. Factor, VIII, Factor IX, or Factor XI). Such proteins or coagulation factors may be used together with proteins and enzymes that are provided by the HHALPC Product (or by ADHLSC Cells or corresponding conditioned cell culture media) and that are involved in the same metabolic pathway or physiological function (e.g. blood coagulation), possibly obtaining synergistic effects. When an HHALPC Product is administered or used in combination with another product, as discussed above, that other product may therefore be such a protein for treating an inherited disease such as a metabolic enzyme or a coagulation factor. Moreover, the pharmaceutical compositions may be generated by cryopreserving HHALPC Product at high concentration (10 million/ml, 50 million/ml, 100 million/ml or more, in appropriate, commercial solutions such as Cryostor) that is thawed and administered to the patients by reconstituting the pharmaceutical composition with an appropriate diluent directly within the cryopreserving vials (without the need of a classified environment and with less logistical requirements).

This approach may provide pharmaceutical compositions that provide longer and/or improved therapeutic effects than the administration of the isolated recombinant protein, as commonly used for treating an enzyme or coagulation factor deficiency. A pharmaceutical composition may therefore comprise (a) an HHALPC Product as described herein, such as HHALPC Progeny or conditioned culture medium thereof, (b) another product as described herein, such as a drug, therapeutic agent, another cell type, or other biological material, more particularly a protein for treating an inherited disease, such as a metabolic enzyme (e.g. Ornithine transcarbamylase or UDP-glucuronosyl transferase 1A1) or a coagulation factor (e.g. Factor, VIII, Factor IX, or Factor XI) and (c) a pharmaceutically acceptable carrier or diluent. At this scope, specific types of HHALPC Products and ADHLSC Cells (such as sub-population, cell lines, and fraction thereof) may be selected during the manufacturing process such as to present the most appropriate level of production for a series of proteins (as absolute values and/or as ratio among such proteins) that are involved in a given metabolic pathway or physiological function (e.g. blood coagulation). For instance, specific sub-populations of HHALPCs or HHALPC Progeny (and related manufacturing process) may be selected such as to provide cell populations (that can be maintained as cell line, deposited cell preparations, or otherwise stored) that have a balanced expression of multiple coagulation factors (e.g. two or more of the extrinsic factors Factor V, Factor VII, and Factor X and/or two or more of the intrinsic factors Factor VIII, Factor XI, Factor XIII, Factor XII, and Factor IX) that is appropriate for one of the different types of blood coagulation deficiency, such as Haemophilia (type A is associated to Factor Deficiency; type B is associated to Factor IX Deficiency; type C is associated to Factor XI Deficiency).

The use of HHALPCs or HHALPC Progeny in general, within compositions and in methods of treatments, can provide therapeutic effects to liver diseases such as those listed above but can be also associated to in vitro studies in substitution of primary hepatocytes or liver cell lines. In particular, HHALPC Progeny can be used in (early) pharmacological and toxicological methods for evaluating the efficacy (if the HHALPC Product expresses a potential drug target for a liver-specific or non-specific disease), the metabolism, the stability, and/or the toxicity of compounds (e.g. biological or chemical entities).

Such in vitro methods and uses should generally comprise the following steps:
(a) Providing a preparation of HHALPC Product (e.g. HHALPCs or HHALPC Progeny in the form of cells, cell extract, or conditioned medium obtained from HHALPCs or HHALPC Progeny);
(b) Exposing said HHALPC Product to one or more exogenous components selected from chemical compounds, proteins, nucleic acids, lipids, sugars, metals, salts, viruses, bacteria, or cells; and
(c) Detecting the effects of said one or more exogenous components on HHALPC Product and/or detecting the presence, localization, or alteration of said one or more exogenous components following the exposure to HHALPC Product.

HHALPCs and HHALPC Progeny can express at high level enzymes and other liver-specific proteins that are known to metabolize most of chemicals that are already registered drugs, candidate drugs still under development and pre-clinical evaluation for liver-specific effects, or any other chemical that is suspected having liver-specific effects that can be undesired (i.e. for an hepatotoxic compound) or desired (if HHALPCs and HHALPC Progeny express an enzyme and other liver-specific protein that is known to be itself a target for candidate drugs for a liver-specific or unspecific disease such as cancer and the compound may be then considered as a candidate drug for such disease).

In general, HHALPC Products in the form of cells, cell extract, or conditioned medium obtained from HHALPCs or HHALPC Progeny can be evaluated in step (c) above for evaluating metabolism, elimination and toxicology of chemicals, inorganic compounds, biologicals, bacteria, viruses, or cells by the analysis of general features such as cell morphology or viability (e.g. in cytotoxicity tests). However, alternative or additional criteria may be included such as the up- or down-regulation of liver-specific (or unspecific) proteins, or any alteration (e.g. degradation, aggregation, activation, or inhibition) of proteins within the HHALPC Product (e.g. HHALPCs, HHALPC Progeny, or cell extract, or conditioned medium obtained from HHALPCs or HHALPC Progeny).

Alternatively (or in combination with the criteria evaluated for the HHALPCs or HHALPC Progeny and derived biological materials), step (c) may involve the analysis on how these one or more exogenous components have been internalized and/or modified or not by HHALPCs or HHALPC Progeny and derived biological materials. These analytical criteria vary according to the type of exogenous components as described in the literature, for example degradation, binding with other proteins, persistence in cell culture, aggregation, infectivity (for viruses), or differentiation or viability (for cells).

The literature on in vitro assays involving cells and derived products (i.e. cell extracts, conditioned media) can provide a guidance on how HHALPCs or HHALPC Progeny in the form of cells, compositions, and derived biological materials (i.e. HHALPC Products) can be used in vitro as indicated in the steps (a)-(c), e.g. regarding concentration, timing, culturing and assay condition, and analytical technologies. Similar assays may be also performed by introducing HHALPCs or HHALPC Progeny in animals, such as non-human animals, in step (a) and then administering one or more exogenous components to the animals in step (b) to determine, in step (c), if and how said one or more components modify HHALPCs or HHALPC Progeny (or related biological materials) and/or are modified by HHALPCs or HHALPC Progeny in these animals.

HHALPC Products, HHALPCs, and HHALPC Progeny in particular, can used for the in vivo (i.e. for therapeutic uses of such cells) and in vitro (e.g. for pharmaco-toxicological uses) methods involving chemicals or biologicals described above within a kit as described above. In particular, the kit can comprise, in addition to such cells (or derived biological materials), further elements that allow using and/or detecting them and their activities when they are exposed to a panel of compounds (resulting from at least one change in the structure, the metabolite, and/or the concentration of the compound to be tested), as well as reference compounds, solutions and/or other cells that would help comparing and evaluating the effects that are observed in assays involving the use of HHALPCs and HHALPC Progeny.

Hence, HHALPCs and HHALPC Progeny can provide in vitro models involving continuous and readily available cells with limited variability in the hepatocyte-like pattern of enzymes stable over time in culture and from batch to batch, in particular as alternative cells to primary hepatocytes in “ADMET” (administration, distribution, metabolism, elimination and toxicology) or cytotoxicity tests (i.e. on hepatocyte viability and/or functional efficiency).

HHALPCs and HHALPC Progeny can be used in methods for testing agents for treating liver infections or for allowing the efficient replication of a virus that infects liver and hepatocytes in particular. HHALPCs and HHALPC Progeny can be differentiated and/or genetically modified before or after exposing to the virus (e.g. a hepatitis virus). Then, the infected cell population can be exposed to a predetermined amount of candidate compound for treating the infection for observing any useful effect (e.g. on viral replication), used for purifying viral particles, or used for assessing any potential in vivo effect of viral infection, as shown for other liver progenitor cells in connection to Hepatitis C infection, liver fibrosis, or carcinogenesis (Wu X et al., 2012; Wang C et al., 2012; Torres D M and Harrison S A, 2012).

The teachings of all references herein specifically referred to are incorporated by reference. The invention will now be illustrated by means of the following examples, which do not limit the scope of the invention in any way.

### EXAMPLES

### Example 1: Analysis of Cell Surface Proteins on HHALPCs

### Materials & Methods

### HHALPCs Isolation and Expansion in Cell Culture

HHALPCs were recovered subsequent to primary culture of the liver parenchymal cellular fraction achieved after two-step collagenase perfusion, filtration and low-speed centrifugation, as described elsewhere (Najimi M et al., 2007), using five distinct human donors. Such liver cells are suspended in a cryopreservation medium that is prepared in ViaSpan Solution and then are maintained in liquid nitrogen by using appropriate vials, bags, or other system for long-term storage and preservation of human cells. The cryopreserved liver cell suspensions are used by quickly thawing them at 37° C. and washing them twice in 10× volume of human albumin 5% supplemented with 2.5 g/L Glucose, 0.084 g/L bicarbonate and 5000 IE/UI/ml Heparin LEO®. After centrifugation at 224 g for 10 minutes at 4° C., the cell pellet is suspended in the required cell culture media.

HHALPCs were cultured on CeIIBIND® flasks (Corning®) in Dulbecco's modified Eagle's medium (DMEM) containing 4.5 g/L glucose (Invitrogen), supplemented with 10% fetal calf serum (Gibco) and 1% penicillin/streptomycin (Invitrogen), at 37° C. in a fully humidified atmosphere (5% CO₂). Upon reaching 80% confluence, cells were lifted with 0.05% trypsin-EDTA (Invitrogen) and re-plated at a density of 5000 cells/cm². The composition of media and buffers can be adapted to the actual requirements for Good Manufacturing Processes by making use of additional or alternative GMP-grade reagents. The viability of the recovered cells was evaluated using the trypan blue dye exclusion method.

### Cell Surface Marker Screening by Flow Cytometry Using BD Lyoplate Technology

The BD Lyoplate™ human cell surface marker screening panel (Cat. No. 560747; BD Biosciences, Heidelberg, Germany) was used to characterize HHALPCs. The kit contains 242 purified monoclonal antibodies to cell surface markers, as well as isotype controls to assessing nonspecific backgrounds. Before use, plates containing lyophilized antibodies were centrifuged at 300 g for 5 minutes. The antibodies were then reconstituted in 110 μl of sterile Dulbecco's Phosphate-Buffered Saline (DPBS).

The assay was performed with HHALPCs that were generated from each of the five donors, according to the manufacturer's instructions. Briefly, ADHLSCs were harvested at passage 5 using 0.05% trypsin-EDTA. After washing in DPBS, cells were resuspended in Pharmingen stain buffer containing 5 mM EDTA at a concentration of 1.25×10⁶ cells/ml. Eighty microliters of cell suspension per well was then transferred to 96-well plates and stained with 20 μl of specific primary antibodies for 30 minutes on ice. Thereafter, the cells were washed twice with Pharmingen stain buffer including 5 mM EDTA and stained with 100 μl of Alexa Fluor 647-labeled anti-mouse or anti-rat secondary antibody (diluted 1:200 in Pharmingen stain buffer including 5 mM EDTA) for 30 minutes on ice. After washing, cells were fixed with BD Cytofix fixation buffer and transferred from the 96-well plates to single BD FACS tubes. Fluorescence was measured with a BD FACS Canto II cytometer on 10,000 cells using FACSDiva software. For analysis, background fluorescence was set manually for each sample based on its appropriate isotype using FlowJo software. Results are expressed as a percentage of positive cells in the population or median fluorescence intensity (MFI).

Characterization of HHALPCs by Flow Cytometry with Other Antibodies

Cells are harvested, suspended at a concentration of 500-1000/μl in PBS buffer (Cat. No. SH30028.03, Thermo Fisher) and incubated for 30 min at 4° C. with the following fluorochrome-labeled antibodies specific for the indicated antigens that are used at the concentration indicated by the manufacturers or according to instructions for the antibodies included in BD Lyoplate. Corresponding control isotype antibodies are used for evaluating non-specific binding of monoclonal antibodies. Cells are then washed and suspended in PBS/BSA for reading with BD Biosciences FACSCanto II Flow Cytometer.

For CXCR4 (CD184) staining, liver cells were first incubated with DPBS-bovine serum albumin (BSA) 1.5% for 20 minutes at 4° C. to prevent nonspecific binding. Next, the cells were washed with DPBS-BSA 1.5% and stained with 5 μl of PE rat anti-human CXCR4/CD184, APC mouse anti-human CD90, or their respective isotypes (BD Biosciences) for 30 minutes on ice. Finally, the cells were washed and fixed using a stabilizing fixative (BD Biosciences). For intracellular staining, liver cells were fixed and permeabilized with 200 μl of cytofix/cytoperm buffer (BD Biosciences) for 20 minutes at 4° C. The cells were then washed with perm/wash buffer and stained with PE rat anti-human CD184 or its isotype diluted in perm/wash for 30 minutes on ice. Next, the cells were washed twice and fixed with stabilizing fixative (BD Biosciences). Fluorescence was measured with a BD FACS Canto II cytometer on 10,000 cells using the FACSDiva software. Data analyses were performed with FlowJo software. Control staining with anti-CD90 antibodies confirmed the correctness of the protocol in these experimental conditions.

### Real-Time PCR

Total RNA was extracted from four donors using TriPure isolation reagent (Roche, Mannheim, Germany), following the manufacturer's instructions. Briefly, 1.5 10⁶ cells were homogenized in TriPure reagent, mixed with chloroform, shaken vigorously for 15 second and centrifuged at 12,000 g for 15 minutes at 4° C. RNA in the upper aqueous phase was precipitated by isopropanol, washed in 75% ethanol, air-dried, and dissolved in RNase-free water. RNA samples were stored at −80° C. after quantification with a NanoDrop 2000 spectrophotometer (Thermo Scientific). The cDNA was synthesized from 1 μg of total RNA by reverse transcription polymerase chain reaction (RT-PCR) using a high-capacity kit (Applied Biosystems). Thereafter, 10 ng of RT product was deposited in each well of a TaqMan® array human extracellular matrix and adhesion molecules (Invitrogen), as instructed by the manufacturer. Plates were read using the Applied Biosystems StepOnePlus real-time PCR system.

### Results

ADHLSC Cells and HHALPCs are cell populations that can be both derived from preparations of cryopreserved human primary liver cells that are produced using normal adult human livers (under non-GMP and GMP conditions, respectively) and that can be differentiated in vitro into cells having hepatocyte-like activity and morphology, with certain markers that are common to ADHLSC Cells, HHALPCs and hepatocytes and other distinguishing HHALPCs as Mesenchymal Stem/Stromal Cells (Najimi M et al., 2007).

However, the therapeutic benefit of Mesenchymal Stem/Stromal Cells (MSCs) such as ADHLSC Cells and HHALPCs for a number of diseases, including liver diseases, highly depends from effects of their GMP preparation on actual level of engraftment. Such cells are known to express at least some of the receptors related to engraftment levels and share a mechanism similar to leukocytes and hematopoietic stem cells to engraft in injured organs, relying on various receptors for rolling, firm adhesion, and transmigration through the endothelium. Cell culture passaging and the conditions may affect how such receptors are actually present and functional in cell preparations for human use.

HHALPCs require specific manufacturing and quality criteria, such as compliance to GMP conditions, improved growth rate and population-doubling level, and compliance to quality specifications prior to cryopreservation and clinical use (i.e. cells must remain viable and undifferentiated, present a given combination of positive/negative markers, while maintaining the capacity to differentiate toward functional hepatocytes). This up-scaled process, that required the optimization of some cell culture parameters, was initially accomplished in multitray stack (e.g. Corning CellStack) and then transferred to multiplate bioreactor (e.g. PALL Xpansion 10), confirming that liver progenitor cells such as HHALPC can be provided at industrial scale, having homogenous quality and quantity (Egloff M et al., 2013). HHALPCs were cultured and expanded for 5 passages in large scale culture conditions on CellIBIND® plastic (treated to facilitate adhesion, in the absence of EGF) before being comprehensively screened at the level of cell surface proteins by flow cytometry, using the BD Lyoplate™ kit, in order to define the effects of GMP preparation on a panel of more than 200 human cell surface markers, covering co-stimulatory molecules, cytokine/chemokine receptors, etc., using HHALPCs that were generated from five different liver donors (Table 1).

The cells from all five donors were measured positive for a series of mesenchymal markers on cell surface, together with adhesion properties, a series of tetraspanins (including CD81, of particular interest since it is responsible of hepatocyte permissiveness to Plasmodium infection; Yalaoui S et al., 2008), an amino acid transporter such as CD98, and only a specific chemokine receptor (CD140b, corresponding to PDGFRbeta) while all other cytokine receptors appear not exposed on HHALPCs cell surface. Other markers including some Adhesion markers (CD54, CD164, CD165, and CD166), Cell surface receptors (CD95), and Complement-related proteins (CD46, CD55, and CD59) were still detected consistently, but at lower levels, across donors (Table 1). The intensity of signals confirmed that HHALPCs express at very low level cell surface markers for other cell lineages (hematopoietic, epithelial, and/or endothelial) such as CD45, CD117, CD34, or HLA-DR. Moreover, a few other MSCs/pluripotency markers were consistently found strongly expressed across all donors, including CD13 and, interestingly, CD105, whose expression on cell surface appears strongly increased after culture on CeIIBIND when compared to ADHLSC Cells (Najimi et al, 2007).

HHALPCs, as ADHLSC cells, did not express major receptors for immune response induction and immune modulation (such as CD1, CD7, CD70, HLA-/-DR, CD27, CD28, CD40, CD80, or CD112) across all donors, confirming their poorly immunogenic phenotype. These cells express at variable level across donors some adhesion markers (including CD26, CD49a, CD49d, CD58, CD61, CD71, CD142, CD146, CD201, CD340, and HLA-A/-B/-C; Table 1). Those of the markers defined above as variably associated to HHALPCs may be considered useful either as positive markers or negative markers when found in HHALPCs, depending on the final use of cells (being the therapeutic use or specific patients).

TABLE 1
General features of HHALPCs as determined by BD Lyoplate.

| Mean Fluorescence | |
|---|---|
| Intensity values in | |
| BD Lyoplate Assay | BD Lyoplate-detected antigen |
| Superior to 750 in all | Mesenchymal or pluripotent markers (CD13, CD73, CD90, |
| samples | and CD105) |
| | Adhesion markers (CD29, CD44, CD47, CD49b, CD49c, |
| | CD49e, and CD147) |
| | Tetraspanins (CD9, CD63, CD81, and CD151) |
| | Cell surface receptors (CD140b) |
| | Immunomodulation (β2-microglobulin) |
| | Transport protein (CD98) |
| Superior to 250 in all | Adhesion markers (CD54, CD164, CD165, and CD166) |
| samples | Cell surface receptors (CD95) |
| | Complement (CD46, CD55, and CD59) |
| Variable across | CD26, CD49a, CD49d, CD58, CD61, CD71, CD142, |
| samples | CD146, CD201, CD340, and HLA-A/-B/-C |

Other cell surface markers that are included in the assay were characterized as being expressed at very low levels or negative, including cell surface markers previously characterized for ADHLSC cells (such as CD34, CD45, CD117, and HLA-DR) or not characterized for ADHLSC cells but rather for immune response (including CD28, CD30, CD200, CD229, CD275, CD279, CD300, and CD357).

HHALPCs can be also measured positive for a series of other markers and activities not related to cell surface proteins. In the perspective of using HHALPCs in specific clinical indications and for optimizing manufacturing process, the initial criteria may be improved by identifying additional markers (being cell surface proteins, secreted proteins, or related to enzymatic activities) that allow characterizing cell quality and optimizing every step of such process (i.e. selection of primary liver cells, cell culture conditions, formulation, storage, and/or patient's selection). Building on this set of data, the additional proteomics-/transcriptomics-based comparison across samples of HHALPCs and human primary hepatocytes may suggest further relevant markers that may be tested in using flow cytometry, ELISA, or other commercial kits, either at the level of single marker analysis or multiple parallel analysis (e.g. by using antibodies for cell surface markers other than those contained in the BD Lyoplate™ kit).

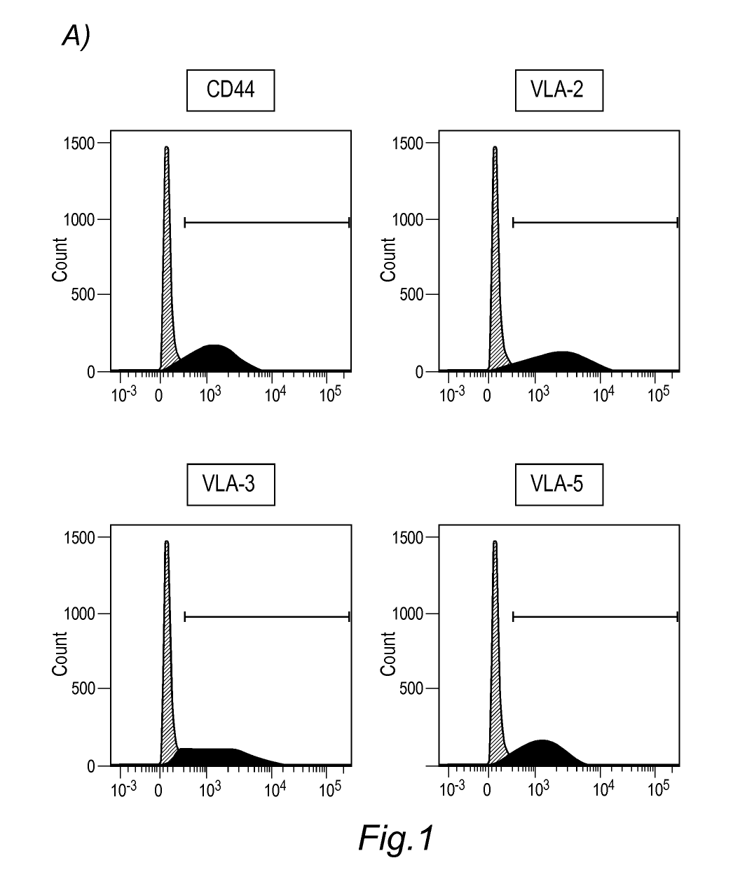

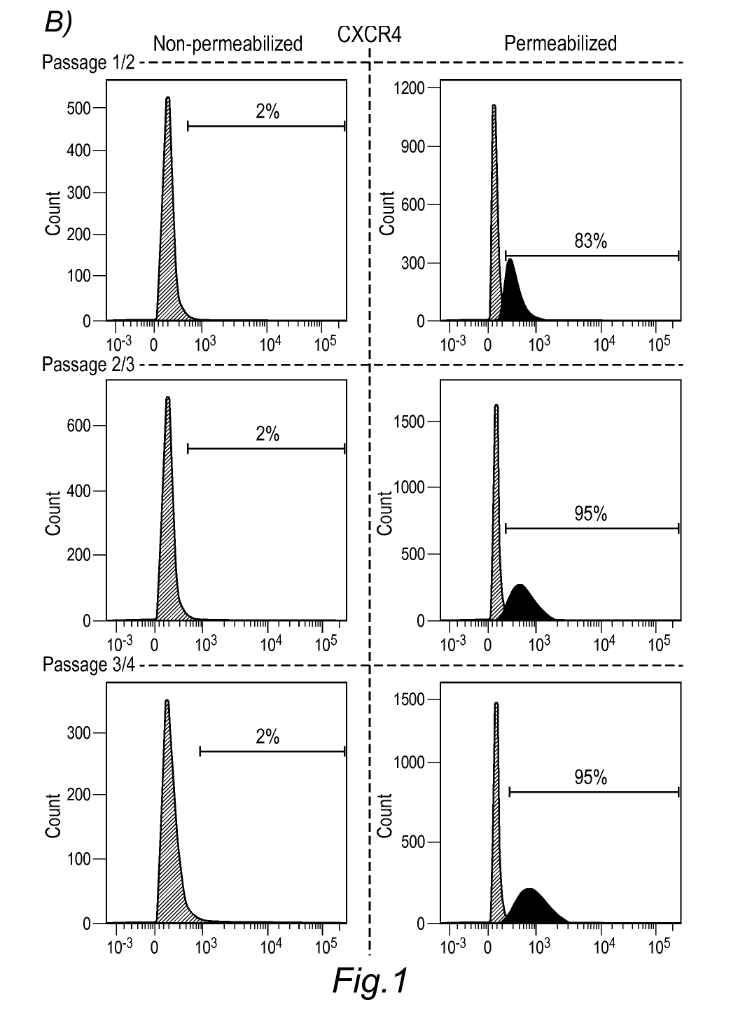

The BD Lyoplate negative data were confirmed at the mRNA level for some markers such as CD162 (PSGL-1), fucosyltranferase IV (SSEA-1), or sialyl-Lewis X (SLeX), a tetrasaccharide component of PSGL-1 required to bind E-selectin on their surface. The absence of such enzymes providing adhesion-relevant sugars to receptors such as CD44 makes such receptors, even if expressed on the cell surface, possibly not functional as an adhesion protein. Other receptors such as VLA-2 (CD49b, binding to collagen) VLA-3 (CD49c, binding to laminin), and VLA-5 (CD49e, binding to fibronectin) are found at high level at mRNA level and by flow cytometry (FIG. 1A). The same observation is not confirmed for VLA-4 (being only its beta subunit CD29 strongly expressed and not its alpha subunit CD49d, or due to manipulation during cell preparation leading to the loss of part of its extracellular domain) and mostly important CXCR4/CD184.

CXCR4 expression is an important protein involved in the engraftment/homing process of HSCs and MSCs. At injury sites, CXCR4 binds released SDF-1, which facilitates cell migration to organs (Marquez-Curtis L A and A Janowska-Wieczorek, 2013). Use of the CXCR4 antagonist AMD3100 during cell infusion was shown to inhibit migration of MSCs to the acutely injured kidneys (Liu N et al., 2013). However, it has been reported that CXCR4 expression decreases rapidly after MSCs isolation and only a very small percentage of cells or none at all express CXCR4 after a few passages (Wynn R F et al, 2004). In fact, in vitro expansion of MSCs induces progressive internalization of CXCR4 as a way for cells to adapt to culture conditions, to the point where there is no CXCR4 remaining on their surface (Pelekanos R A et al., 2014). Surface expression of CXCR4 has therefore been evaluated at each passage by flow cytometry and it has been found that all donors tested showed a very low surface receptor expression when cells are not permeabilized. However, when the cells were permeabilized, a large proportion of the cell population expressed CXCR4, suggesting that a large portion of the population had already started to internalize CXCR4 (FIG. 1B).

Some research groups have decided to induce externalization of CXCR4 on the surface of MSCs, a key point to enhance MSC homing. Different methods have been used to upregulate CXCR4, such as culturing in presence of valproic acid (Gul H et al., 2009), SDF-1 (Jones G N et al., 2012), or a cytokine cocktail (Shi M et al., 2007). However, neither the cytokine cocktail, nor pre-incubation with SDF-1 appears having any effect on CXCR4 externalization, leaving the other opportunities for developing HHALPCs with improved engraftment properties.

The strong positivity of HHALPC for a restricted number of cell surface markers across different protein categories allows using antibodies such as anti-CD140b, anti-CD105, anti-CD9, anti-CD47, anti-CD49c, anti-CD49e, anti-CD29, anti-CD147, anti-CD73, anti-CD81, anti-CD151, and/or anti-CD98 for assessing the quality, purity, and/or identity of HHALPCs during their manufacturing and/or prior to their use. Together with the other criteria that are listed in the literature (Najimi M et al., 2007) and (if available) the clinical information on the human subject who has provided the initial primary liver cells preparation, the detection of cell surface markers listed above may allow further optimizing the most appropriate therapeutic use and/or the human subject for administering HHALPCs.

The findings obtained by using BD Lyoplate in distinct HHALPCs preparations from different donors provide a guidance for identifying which additional markers and biological activities can be associated to HHALPCs and then improving their GMP manufacturing, as well as their in vitro or in vivo uses. However, the pattern of expression of some adhesion proteins that are important for HHALPCs engraftment may result from the culture process. The identification of multiple cell surface markers would help determining which GMP cell culture conditions can improve cell engraftment and thus improving HHALPCs suitability for medical uses requiring the repopulation of human liver with hepato-active cells (such in certain inborn metabolic liver disorders or acute/traumatic major liver injuries, or as alternative to liver transplantation), as well as the possibility to use such cells for delivering systemically enzymes, growth factors, and other proteins that are either naturally expressed by functional hepatocytes (such as those related to coagulation, cirrhosis, or fibrosis, in case of patients affected by related disorders) or non-hepatic proteins that are appropriately expressed by genetically modified HHALPCs (such as antibodies or hormones that may be useful in a treatment of a large variety of indications such as cancer, diabetes, or inflammatory disorders). Additional preclinical models and approaches for validating the administration of HHALPCs with respect to liver repopulation and regeneration have been reviewed in the literature (see the book “Liver Regeneration Basic Mechanisms, Relevant Models and Clinical Applications”, Edit.: Udayan M. Apte, Elsevier 2015).

### Example 2: Validation of HHALPCs Therapeutic Properties

### Materials & Methods

### HHALPCs Preparation and Administration to Patients Affected by Urea Cycle Disorders

HHALPCs were produced from healthy human liver cell suspensions and expanded in five passages, as indicated in Example 1, and then harvested, cryopreserved in CryoStor-10 (10% dimethyl-sulphoxide), and stored in liquid nitrogen. Before use, HHALPC were thawed and washed in albumin solution, and then formulated in an aseptic environment in a GMP facility as a cell suspension containing 250×10⁶ cells in 0.084 sodium bicarbonate, 5% human albumin, and 500 IU heparin in a 50-ml plastic bag. HHALPC were infused intravenously via a percutaneous transhepatic portal catheter, that was inserted under general anaesthesia by direct transhepatic puncture of the right/left portal vein to the main portal vein at the splenomesenteric confluent, under radiologic and ultrasound guidance, at a flow rate of 0.5-2 mL/min. Each infusion was performed under moderate anticoagulation treatment with bivalirudin (Stephenne X et al., 2012) and followed by the concomitant therapies (immunosuppressive treatments and regular treatment of each patient for the Urea Cycle disorder).

Patients were monitored according to standard protocols for such disorders. Additionally, in vivo ureagenesis was assessed using stable non-radioactive isotopes to evaluate actual urea cycle activity by measuring the incorporation of ¹³C into urea into plasma from a ¹³C-labelled precursor that patients ingested in the form of sodium acetate, as described in the literature (Yudkoff M et al., 2010).

### HHALPCs Preparation and Administration to Patients Affected by Hemophilia

HHALPCs were produced as described in Example 1 and above but were in part radiolabelled prior to their final formulation and administration using 111-Indium (¹¹¹In). Briefly, 25 million HHALPC were suspended in 5 mL NaCl 0.9%, incubated 15 minutes at room temperature with ¹¹¹In-DTPA at a concentration of 20 μCi/1.10⁶ cells under gentle shaking. Cells were then washed, the labeling efficiency was measured with a dose calibrator (Capintec Radioisotope Calibrator CRC12) and calculated as follows: [Radioactivity from cells]/[Radioactivity from (supernatant+Cells)]×100. The labeling efficiency was estimated at 79%.

HHALPC (radiolabeled or not) were formulated in 5% Human albumin (Hibumine, Baxter) supplemented with glucose (0.025 g/L, Stereop), 6.5 mg/mL Sodium Bicarbonate (B52 Braun), 10 UI/mL Heparin (LeoPharma) and 0.78% Lysomucyl (Zambon). HHALPC were infused via a peripheral intravenous catheter placed in the forearm, with one initial infusion of 25 million cells labelled with indium, followed by four infusions of 250 million cells every 2 weeks. Clinical monitoring of cardio-respiratory and coagulation parameters was done during and after cell infusion. During this infusion period, both prophylactic treatment with recombinant Factor VIII and standard immunosuppression (with methylprednisolone and Tacrolimus) were performed. Dosing of blood Factor VIII level and coagulation profile, including thromboelastogram, were taken as biochemical response assessment parameters. Factor VIII requirement and clinical bleeding characteristics of the patient were evaluated.

Dynamic acquisition during the entire duration of infusion and a total body imaging acquisition at indicated time points after cell infusion were performed by SPECT imaging. The hepatic retention of ¹¹¹In-DTPA signal was calculated as the ratio of regions of interest to whole body uptake with PMOD analysis program.

### Results

HHALPC administration represents a therapeutic solution for a series of inherited or acquired disorders requiring the reconstitution of damaged liver tissues (for example in case of a chronic or acute insult due to viral infection, exposure to toxic compounds, fibrotic disorders, or cancer) or liver cells expressing functional proteins that exert their activities at intracellular level (e.g. for metabolic functions) or in extracellular compartments (e.g. as secreted proteins exerting immunomodulatory activities within liver tissues or other activities in tissues where such proteins are transported by blood circulation). Depending from the disorder and status of the patients, HHALPC may be prepared, formulated, and administered using distinct, appropriate approaches.

The therapeutic usefulness of HHALPC has been tested in clinical settings, demonstrating that HHALPC is a cell therapy product with multiple properties of interest and suitable for different modes of administration and indications.

As a first example, HHALPC administration can increase ureagenesis in patients suffering from Urea Cycle Disorders, inherited metabolic diseases associated with significant medical complications and with treatments that are limited and palliative, imposing heavy burden to patients and families. Cell-based therapies can provide sufficient metabolic liver functions for attenuating the clinical course, at least until allogenic liver transplantation becomes feasible.

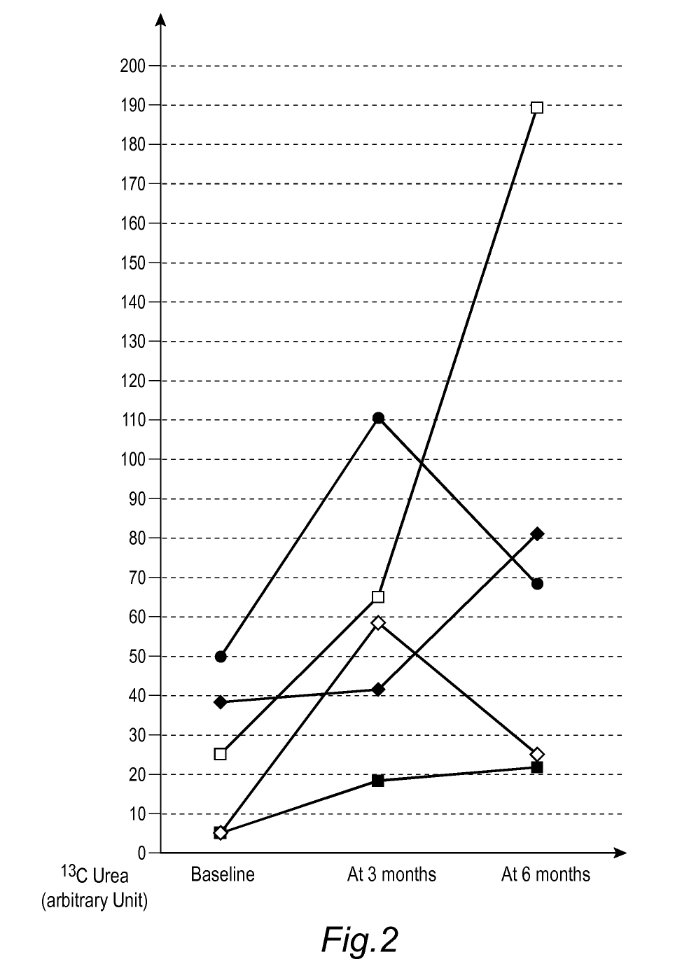

GMP-produced pharmaceutical compositions comprising well-characterized HHALPC can be infused via the portal route. In a first study involving paediatric patients with different diseases, weight, and age, HHALPC were administered at different dosages (12.5×10⁶ to 200×10⁶ cells/kg, in a variable number of infusions over 1-4 days), measuring a series of metabolic and safety criteria over several months. In particular, the metabolic effect of HHALPC on urea cycle functionality was evaluated by measuring the in vivo ureagenesis using a labelled urea precursor. This disease-related biological activity appears positively affected by HHALPC administration, which is tolerated by patients already under chronic, long-term supportive treatments, like nitrogen scavengers (FIG. 2).

A further example is Hemophilia A, a X-linked bleeding disorders caused by a deficiency of coagulation factor VIII that is consequently administered to patients by prophylactic, periodical intravenous injections. Such current standard of care is associated with development of neutralizing anti-factor VIII antibodies in several patients, with compromised efficacy and increased cost of treatment (see Kabel A, 2014 for a review on bleeding disorders and their therapeutic management). Cell-based treatments that allow providing Factor VIII, endogenously and in a localized manner, may provide patients with longer durations of response with lesser complications are ongoing. Since liver itself is a major site of factor VIII synthesis and mesenchymal stem cells have been shown to control bleeding in animal models of haemophilia, progenitor cells of hepatic origin that engraft in human liver and are poorly immunogenic like HHALPCs may be used for providing Haemophilia A patients with Factor VIII at least for reducing the administration of recombinant, exogenous Factor VIII.

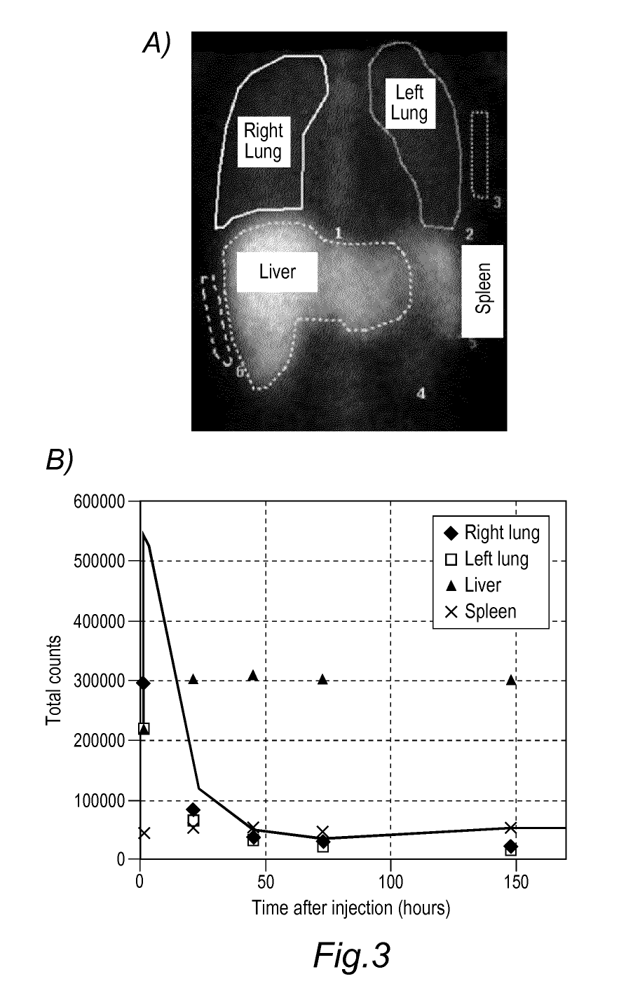

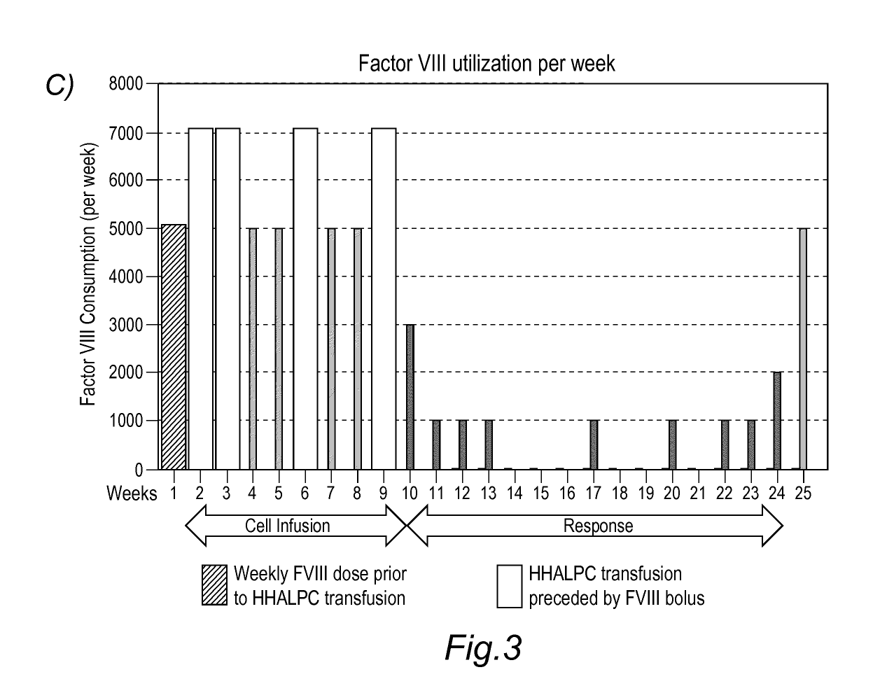

A patient suffering from severe haemophilia A with recurrent episodes of haemarthrosis that cause right ankle disability (despite prophylactic injection of Factor VIII at high dosage) was treated by intravenous administration of HHALPC. This clinical intravenous infusion of cells naturally expressing Factor VIII like HHALPC was performed in parallel to regular Factor VIII administration, and followed at the level of both bio-distribution in relevant tissues and of analysis and effect on Factor VIII needs by the patient (FIG. 3). Imaging during infusions showed that labelled HHALPC were initially trapped in the lungs but then quickly, within 1 hour, the cells were also detected in the liver at a level well superior to lungs and spleen. Interestingly, at 4 hours after infusion, HHALPC could be also detected in the right ankle, which was the site of repeated haemarthrosis in the patient, suggesting that HHALPCs may provide a potential relief also to this disorder. Indeed, patient's Factor VIII requirement drastically dropped in the 15 weeks following the end of HHALPCs injections, with Factor VIII that was injected only when there was a bleeding episode. During this response period, the biochemical markers did not show any significant change but the patient observed much less bleeding episodes while undertaking physical activities even without prior prophylactic Factor VIII infusion, with a single episode of hemarthrosis for which he needed 1000 IU of Factor VIII for resolution, while in general he would have injected 5000 IU for the same result. He had a subjective feeling of being able to undertake more rigorous physical activity without bleeding episodes, in the absence of prophylactic factor VIII injection.

Thus, HHALPC provide a drug product that can be used according to different regimens, formulations, and clinical settings for achieving a therapeutic effect related not only to metabolic liver-related activities but also to the secretion of proteins such as Factor VIII (or other proteins) that can exert coagulant or immunomodulatory effects in different locations, for example within articulations, reducing the use of other drugs targeting such locations, directly or indirectly.

In a further example, the intravenous administration of HHALPC was performed in patient suffering from Urea Cycle Disorders, to monitor the tolerance and potential side effect and to explore the distribution of HHALPC in the liver after post-infusion. Batches of HHALPC were produced in GMP conditions and administered to a patient suffering from OTC deficiency, with elevated ammonia and glutamine blood levels combined with low arginine and citrulline blood levels.

The patient received 940×10⁶ of progenitor cells (16.3×10⁶ cells/Kg body weight) (235×10⁶ cells per administration). The viability of cells was assessed just after reconstitution, and ranged between 84% and 88%. HHALPC were infused intravenously through a peripheral catheter. Intravenous glucose was administered during each infusion procedure in parallel to Bivalirudin (1.75 mg/Kg/h) as a preventive measure of thrombosis. ACT measures (Activated Coagulation Time measured on fresh whole blood) were collected at each infusion stage but no abnormal ACT value was recorded during the infusions (all ACT values below 350 sec.). Immunosuppression treatment given to the patient included Evérolimus (Certican) with a daily dose of 1.5 mg.

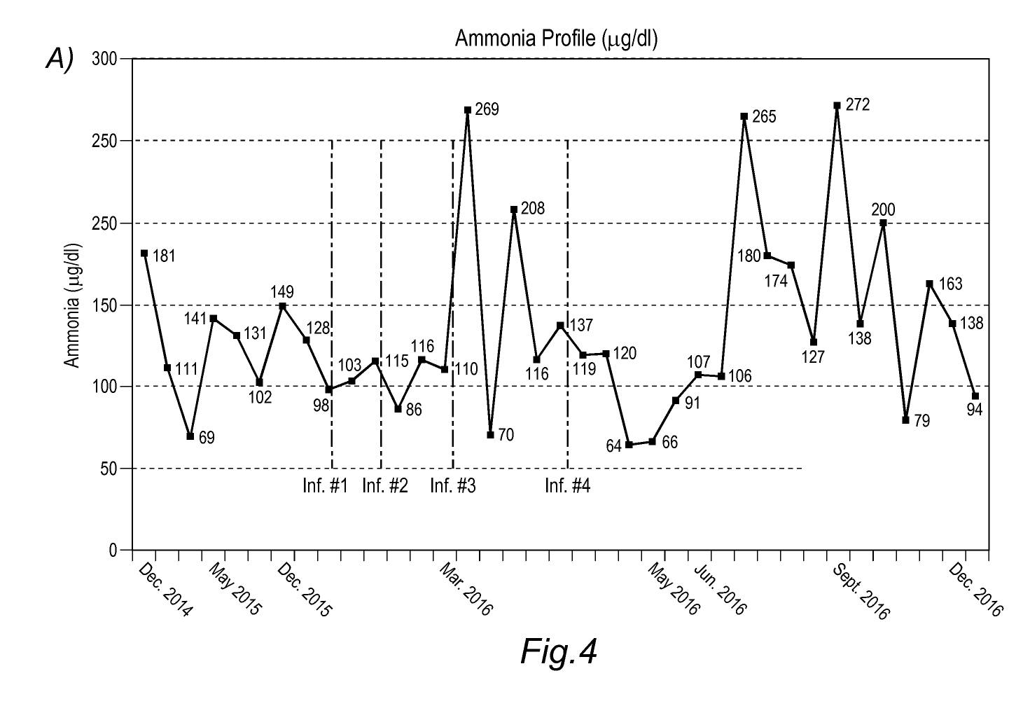

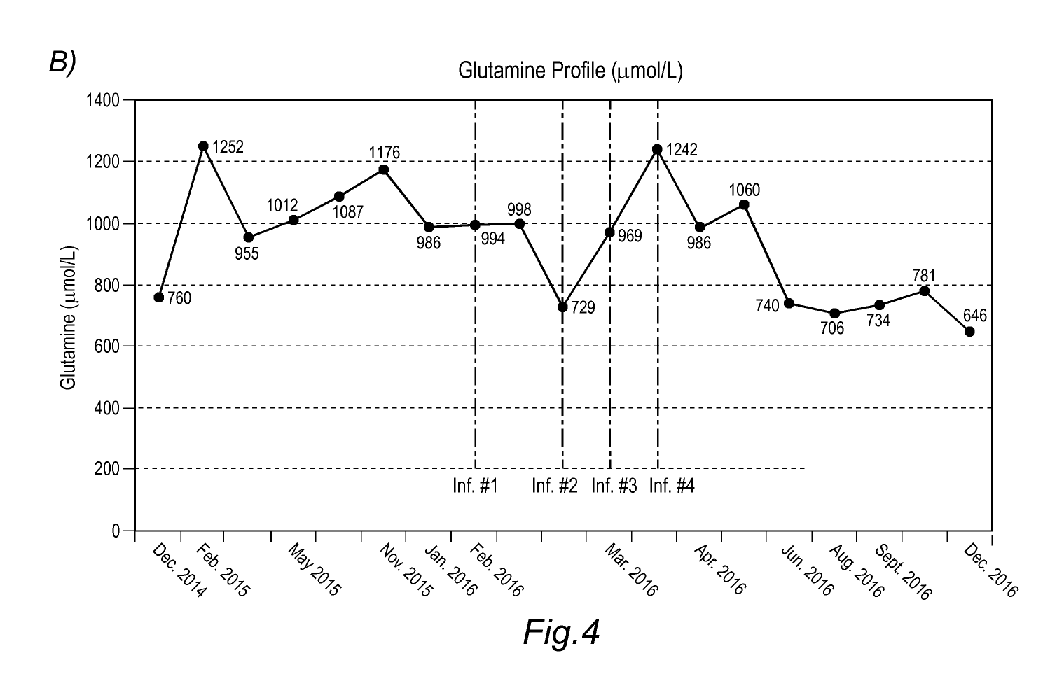

Just after the infusions period, ammonia blood levels were stable during a 2-month period. Thereafter, the patient tended to display higher plasma levels of ammonia but glutamine blood levels were normalized for longer period (FIG. 4). Clinical evaluation following cell infusions revealed some clinical improvements. Five months post-first infusion, the patient was described by the investigator as more dynamic, reactive with a decrease of fatigue episodes reported by the patient herself.

Thus, clinical improvements due to HHALPC-based treatments were shown for different pathologies and using different methods of administration, regimens and dosages.

### REFERENCES

Alépée N et al., ALTEX (2014). 31:441-77.
Azuma H et al., Hepatology (2003). 37: 1385-94.
Bale S et al., Exp Biol Med (2014). 239:1180-91.
Berardis S et al., PLoS One (2014), 9:e86137.
Berardis S et al., World J Gastroenterol (2015). 21: 742-758.
Bieback K. Transfus Med Hemother (2013). 40:326-35.
Caralt M et al., Organogen (2014). 10: 250-9.
Cheng H et al., Biomaterials (2012). 33:5004-12.
Christ B et al., Trends Mol Med (2015). 21: 673-686.
Cigognini D et al., Drug Discov Today (2013). 18: 1099-108.
Dan Y Y, Methods Mol Biol (2012). 826: 11-23.
Darwiche H and Petersen B E, Prog Mol Biol Transl Sci (2010). 97: 229-49.
Ebrahimkhani M et al., Adv Drug Deliv Rev (2014). 69-70:132-57.
Egloff M et al., BMC Proceedings (2013) 7(Suppl 6):P6.
Forbes S et al., J Hepatol (2015) 62(1 Suppl): S157-169.
Gerets H H et al., Cell Biol Toxicol (2012). 28: 69-87.
Gomez-Lechon M I et al., Methods Mol Biol (2012). 806: 87-97.
Gul H et al., Stem Cells Dev (2009)18:831-8.
Herrera M B et al., Stem Cells (2006). 24: 2840-50.
Hook L A, Drug Discov Today (2012). 17: 336-42.
Ibars E et al., World J Gastroenterol (2016). 22: 874-886.
Jones G N et al., Stem Cells Transl Med (2012). 1:70-8.
Kabel A, Int J Hematol Dis (2014). 1: 22-26.
Kavanagh D et al., Stem Cell Rev (2014). 10:587-99.
Khuu D N et al., Cell Transplant (2011). 20: 287-302.
Kim J Y et al., Biomol Ther (2015). 23(6):517-24.
Lancaster M A et al., Science (2014). 345:1247125.
Lee J et al., Biomacromol (2014). 15: 206-18.
Lin C et al., Expert Opin Drug Discov (2015). 10:519-40.
Liu N et al., J Cell Biochem (2013). 114:2677-89.
Maerckx C et al., World J Gastroenterol. (2014). 20:10553-63.
Marquez-Curtis L A and A Janowska-Wieczorek, Biomed Res Int (2013):561098.
Massie I et al., Tissue Eng Part C Methods (2011). 17: 765-74.
Muscari C et al., J Biomed Sci. (2013). 20:63.
Nagase K et al., Biomacromolecules (2015). 16:532-40.
Najar M et al., Int Immunopharmacol (2013). 15: 693-702.
Najimi M et al., Cell Transplant (2007). 16: 717-28.
Nash M et al., Stem Cell Rev (2013). 9: 148-57.
Pelekanos R A et al., BMC Cell Biol (2014) 15:15.
Raicevic G et al., 2015. Cytotherapy. 17: 174-85.
Sahin M B et al., Liver Transpl (2008). 14: 333-45.
Sana G et al., Cell Transplant (2014). 23:1127-42.
Santamaria E et al., Methods Mol Biol (2012). 909: 165-80.
Sarkar D et al., Blood (2011). 118:e184-91.
Scheers I et al., Cell Transplant (2012). 21: 2241-55.
Schmelzer E et al., J Exp Med (2007). 204: 1973-87.
Shi M et al., Haematologica (2007) 92:897-904.
Shiojiri N and Nitou M, Methods Mol Biol (2012). 826: 3-10.
Sison-Young R et al., Toxicol Sci (2015). 147:412-24.
Slany A et al., J Proteome Res (2010). 9: 6-21.
Stephenne X et al., PLOSone (2012). 7: e42819.
Tanaka M and Miyajima A, Methods Mol Biol (2012). 826: 25-32.
Torres D M and Harrison S A, Hepatology (2012). 56: 2013-5.
Wan X et al., Glycobiology (2013). 23:1184-91.
Wang C et al., Hepatology (2012). 55: 108-20.
Wu X et al., PLoS Pathog (2012). 8: e1002617.
Wynn R F et al., Blood (2004) 104:2643-5.
Yalaoui S et al., PLoS Pathog (2008). 4:e1000010.
You J et al., ACS Nano (2013). 7:4119-28.
Yu J et al., PLoS One (2012). 7: e35230.
Yudkoff M et al., Mol Genet Metab (2010). 100 suppl. 1: S37-41.
Zeng W et al., Sci Rep (2015). 5:11100.

1. An isolated human adult liver progenitor cell characterized in that said cell is measured positive for:
(a) the mesenchymal or pluripotent markers CD13, CD73, CD90, and CD105;
(b) the adhesion markers CD29, CD44, CD47, CD49b, CD49c, CD49e, and CD147;
(c) the tetraspanins CD9, CD63, CD81, and CD151; and
(d) CD98, CD140b, and β2-microglobulin.

2. The cell of claim 1 characterized in that the cell is measured positive for:
(a) at least one marker selected from adhesion markers CD54, CD164, CD165, and CD166; and/or
(b) at least one marker selected from CD46, CD55, CD59, and CD95.

3. The cell of claim 1 characterized in that the cell is measured positive for at least one marker selected from CD26, CD49a, CD49d, CD58, CD61, CD71, CD142, CD146, CD201, CD340, and HLA-A/-B/-C.

4. The cell of claim 1 characterized in that the cell is measured negative for at least one marker selected from
(a) CD26, CD49a, CD49d, CD58, CD61, CD71, CD142, CD146, CD201, CD340, and HLA-A/-B/-C; and/or
(b) one or more of CD45, CD117, CD34, and HLA-DR.

5. The cell of claim 1 characterized in that the cell is further measured:
(a) positive for at least one hepatic marker selected from albumin, HNF-4, and CYP3A4;
(b) positive for at least one mesenchymal marker selected from Vimentin, a-smooth muscle actin (ASMA); and
(c) negative for cytokeratin-19 (CK-19).

6. The cell of claim 1 characterized in that the cell is measured:
(a) positive for CD13, CD73, CD90, CD105, CD29, CD44, CD47, CD49b, CD49C, CD49e, CD147, CD9, CD63, CD81, CD151, CD98, CD140b, β2-microglobulin, CD54, CD164, CD165, CD166, CD46, CD55, CD59, CD95, Albumin, and Vimentin; and
(b) negative for CD45, CD117, CD34, and HLA-DR, and Cytokeratin-19.

7. An isolated cell population comprising at least 60% or between 60% and 99% or between 70% and 90% of the cells of claim 1.

8. The cell population of claim 7, wherein said cell population is differentiated into cells presenting liver-specific activities.

9. The cell population of claim 7, wherein said cell population is modified by means of one or more chemical agents, cell culture medium, growth factors, and/or nucleic acid vectors.

10. A biological material isolated from the cell of claim 1, wherein the biological material is formulated with a conditioned cell culture media, a protein extract, a membrane vesicle, or any fraction thereof comprising one or more isolated proteins, nucleic acids, metabolites, and/or antigens.

11. A composition comprising the cell of claim 1.

12. A method of treating a liver disease comprising administering the cell of claim 1 to a subject in need thereof.

13. A method of treating inherited blood coagulation disorders comprising administering the cell of claim 1 to a subject in need thereof.

14. A method for evaluating the efficacy, the metabolism, the stability, and/or the toxicity of one or more compounds, said method comprising:
(a) providing the cell of claim 1;
(b) exposing said cell to one or more compounds; and
(c) detecting the effects of said one or more compounds on said cell, and/or detecting the presence, localization, or modification of said one or more compounds following the exposure to said cell.

15. (canceled)

16. A kit comprising the cell of claim 1.

17. A composition comprising the biological material according to claim 10.

18. A method of treating a liver disease or treating inherited blood coagulation disorders comprising administering the composition of claim 17 to a subject in need thereof.

19. A method for evaluating the efficacy, the metabolism, the stability, and/or the toxicity of one or more compounds, said method comprising:
(a) providing the biological material of claim 10;
(b) exposing said biological material to one or more compounds; and
(c) detecting the effects of said one or more compounds on said biological material, and/or detecting the presence, localization, or modification of said one or more compounds following the exposure to said biological material.

20. A method for evaluating the efficacy, the metabolism, the stability, and/or the toxicity of one or more compounds, said method comprising:
(a) providing the composition of claim 11;
(b) exposing said composition to one or more compounds; and
(c) detecting the effects of said one or more compounds on said composition, and/or detecting the presence, localization, or modification of said one or more compounds following the exposure to said composition.
